SEQUENCE LISTING (19 sequences)

SEQ ID NO: 1 - DNA, 20 bp, Homo sapiens
  source 1..20: mol_type = genomic DNA
actgatttaaacattacttg

SEQ ID NO: 2 - DNA, 20 bp, Homo sapiens
  source 1..20: mol_type = genomic DNA
aactgatttaaacattactt

SEQ ID NO: 3 - DNA, 20 bp, Homo sapiens
  source 1..20: mol_type = genomic DNA
taactgatttaaacattact

SEQ ID NO: 4 - DNA, 20 bp, Homo sapiens
  source 1..20: mol_type = genomic DNA
ttaactgatttaaacattac

SEQ ID NO: 5 - DNA, 20 bp, Homo sapiens
  source 1..20: mol_type = genomic DNA
gttaactgatttaaacatta

SEQ ID NO: 6 - DNA, 20 bp, Homo sapiens
  source 1..20: mol_type = genomic DNA
ggttaactgatttaaacatt

SEQ ID NO: 7 - DNA, 20 bp, Homo sapiens
  source 1..20: mol_type = genomic DNA
tggttaactgatttaaacat

SEQ ID NO: 8 - DNA, 20 bp, Homo sapiens
  source 1..20: mol_type = genomic DNA
ctggttaactgatttaaaca

SEQ ID NO: 9 - DNA, 20 bp, Homo sapiens
  source 1..20: mol_type = genomic DNA
tctggttaactgatttaaac

SEQ ID NO: 10 - DNA, 20 bp, Homo sapiens
  source 1..20: mol_type = genomic DNA
gtctggttaactgatttaaa

SEQ ID NO: 11 - DNA, 20 bp, Homo sapiens
  source 1..20: mol_type = genomic DNA
tgtctggttaactgatttaa

SEQ ID NO: 12 - DNA, 20 bp, Homo sapiens
  source 1..20: mol_type = genomic DNA
gtgtctggttaactgattta

SEQ ID NO: 13 - DNA, 20 bp, Homo sapiens
  source 1..20: mol_type = genomic DNA
tgtgtctggttaactgattt

SEQ ID NO: 14 - DNA, 20 bp, Homo sapiens
  source 1..20: mol_type = genomic DNA
ctgtgtctggttaactgatt

SEQ ID NO: 15 - DNA, 20 bp, Homo sapiens
  source 1..20: mol_type = genomic DNA
cctgtgtctggttaactgat

SEQ ID NO: 16 - DNA, 20 bp, Homo sapiens
  source 1..20: mol_type = genomic DNA
acctgtgtctggttaactga

SEQ ID NO: 17 - DNA, 20 bp, Homo sapiens
  source 1..20: mol_type = genomic DNA
cacctgtgtctggttaactg

SEQ ID NO: 18 - DNA, 20 bp, Homo sapiens
  source 1..20: mol_type = genomic DNA
acacctgtgtctggttaact

SEQ ID NO: 19 - DNA, 20 bp, Homo sapiens
  source 1..20: mol_type = genomic DNA
aacacctgtgtctggttaac

(19) United States
(12) Patent Application Publication
(10) Pub. No.: US 2026/0022383 A1
(43) Pub. Date: Jan. 22, 2026

(54) SUBPOPULATION-DIRECTED REMEDIATION OF DISEASE-ASSOCIATED GENE PRODUCTS

(71) Applicant: Ball State University, Muncie, IN (US)

(72) Inventors: Philip J. Smaldino, Yorktown, IN (US); James P. Vaughn, Pfafftown, NC (US); Yuh-Hwa Wang, Charlottesville, VA (US); Roger S. Cubicciotti, Winston-Salem, NC (US)

(73) Assignee: Ball State University, Muncie, IN (US)

(21) Appl. No.: 19/274,056

(22) Filed: Jul. 18, 2025

Related U.S. Application Data
(60) Provisional application No. 63/673,330, filed on Jul. 19, 2024.
(60) Provisional application No. 63/715,863, filed on Nov. 4, 2024.
(60) Provisional application No. 63/722,879, filed on Nov. 20, 2024.

(51) Int. Cl. C12N 15/113

(52) U.S. Cl. CPC C12N 15/1137; C12N 15/113; C12N 2310/14; C12N 2310/531; C12N 2320/34

(57) ABSTRACT

The present invention relates to compositions and methods for subpopulation-specific modulation of cell function and for treating repeat expansion disorders comprising genetic, degenerative, neurological and cellular diseases, including immune disorders. Also provided are research kits for subpopulation-specific modulation of protein activity and the corresponding potential to discover novel therapeutic compositions. More specifically, the disclosed compositions and methods selectively up- or downregulate at least a first population of a cellular protein or therapeutic target with minimal or negligible effect on the activity of at least a second population of the cellular protein or therapeutic target.

### CROSS-REFERENCE TO RELATED APPLICATIONS

This application claims the benefit of priority to U.S. provisional patent application Ser. No. 63/722,879, filed Nov. 20, 2024, U.S. provisional patent application Ser. No. 63/715,863, filed Nov. 4, 2024, and U.S. provisional patent application Ser. No. 63/673,330, filed Jul. 19, 2024, each of which is incorporated herein by reference.

### REFERENCE TO A SEQUENCE LISTING

This application refers to sequences disclosed in a xml file named “125211000004.xml” of 20,480 bytes, created Jul. 17, 2025, incorporated by reference.

### FIELD OF THE INVENTION

The present invention relates to compositions and methods for selectively modulating cell function at the gene expression, protein isoform and cellular subcompartment level and the use of these compositions and methods in research products, kits and therapeutic preparations, e.g., for the treatment of neuromuscular dysfunction and repeat expansion disorders such as certain genetic, cellular, immune, degenerative and oncogenic diseases. Broadly speaking, the invention is drawn to molecular and cellular remediation—the tuning and alignment of intracellular molecular regulatory networks. More specifically, the disclosed compositions and methods preferentially up- or downregulate at least one population of an endogenously occurring intracellular gene product, e.g., a cellular protein or corresponding RNA transcript, in a subpopulation-specific manner, advantageously providing enhanced specificity for research, preventive and therapeutic applications.

### BACKGROUND OF THE INVENTION

This invention was inspired by the work of coinventor Vaughn and colleagues (JBC (2005) 280:38117-38120) in discovering and characterizing the enzyme G4 Resolvase 1 (G4R1, aka RHAU and DHX36 gene product), a DNA/RNA helicase with unusual biophysical, biochemical and biological properties that are in some ways surprisingly unique for the DEXH helicase class of proteins. Important regulatory roles for G4R1 in both RAN translation and Huntington's disease were first reported by coinventor Smaldino and colleagues (Tseng, et al., JBC 2021 August; 297(2):100914), which indicated the potential of a pathogenic role of this helicase in amyotrophic lateral sclerosis (ALS). Given the relatively ubiquitous presence of G4R1 in mammalian cells and its increasingly apparent distribution across nonmammalian genera. it is not surprising to find that it has rapidly gained attention over the past decade as a potential link in the etiology of a diverse array of human diseases and an important constituent of RNA processing regulatory networks whose integrity is critical to cellular and biological homeostasis. A distinguishing attribute of this class of G4-helicases is the existence of cytoplasmic and nuclear variants (Spliceoforms, isoforms or subpopulations), which offer specificity advantages in probing for therapeutic targets across a wide range of cellular disorders and human diseases (e.g., cellular, genetic, degenerative, immune and oncogenic/neoplastic conditions), including repeat expansion disorders. The instant invention addresses challenges associated with repeat expansion disorders with particular attention to modulating endogenous nucleic acid-binding proteins (NABPs), advantageously catalytic NABPs, comprising helicases, G4-helicases and resolvases as practical targets for understanding, preventing and treating conditions resulting from aberrant RAN translation of toxic peptides implicated in human and animal diseases. The invention is exemplified by experimental work on neurodegenerative diseases in humans and animal models of human disease. While the involvement and significance of protein subspecies and receptor subtypes in human disease have been recognized for decades (e.g., Cubicciotti, U.S. Pat. No. 4,619,895; Bristow et al. (1982) Mol Pharmacol 21:671-679), it has only recently become possible to imagine systematically applying a platform approach to targeting subcellular isoforms of regulatory proteins such as G4R1, FUS and TDP-43 to the discovery, development and delivery of isoform-specific therapeutic products for a wide variety of diseases (cf. Provisional Patent Application Ser. No. 63/673,330 filed on Jul. 19, 2024, and Provisional Patent Application Ser. No. 63/715,863 filed on Nov. 4, 2024, and Provisional Patent Application Ser. No. 63/722,879 filed on Nov. 20, 2024).

G4 helicase enzymes are NABPs that regulate transcription and translation within cells via unwinding quadruplex (G4) DNAs and RNAs and other types of nucleic acid structures (e.g. double helices, hairpins, triplexes, cruciforms, i-motifs, R-loops, pseudoknots and the like). These enzymes are often important regulators of cell division, gene expression and cell cycle progression and are therefore also important cancer drug targets. Intracellular drug targets, including these helicase enzymes and other proteins, often exist within distinct populations in a cell. Drug targeting of proteins for the treatment of neurodegenerative diseases such as FTD and ALS as well as a significant number of other cellular, genetic, degenerative and oncogenic diseases often indiscriminately targets all populations of a protein within a cell. Such indiscriminate targeting may result in the diminished activity of the intended target population but may also result in the diminished activity of other populations of the protein which may be innocuous or have desirable traits. Thus, a need exists for a method and composition for selectively reducing activity of at least one population of an intracellular therapeutic target, while refraining from targeting other populations of the same therapeutic target.

NABPs are proteins that interact with DNA or RNA to regulate gene expression and other cellular activities. In general, DNA-binding proteins (DBPs) regulate gene expression, single strand DNA binding and separation, chromatin formation, certain aspects of epigenetic gene expression and cell development. RNA-binding proteins (RBPs), by contrast, regulate post-transcriptional gene expression, alternative splicing and translation. NABPs are important for many processes, including transcriptional regulation, protein synthesis, and immune response activation. Understanding the functions of NABPs can help improve understanding of gene expression and the pathogenesis of diseases. NABPs can be identified and characterized by experimental techniques, such as pull-down assays, yeast one-hybrid and two-hybrid systems, and electrophoretic mobility shift assays. However, computational approaches, molecular modeling and prediction tools can also be used to identify key regulatory NABPs.

Because NABPs are essential for cellular processes ranging from gene expression to DNA replication and RNA processing, dysregulation of these proteins can lead to a number of cellular disorders. Examples of NABPs implicated in cellular disorders include the RBPs TDP-43, FUS and TIA-1 and the DNA-binding proteins p53, BRCA1, BRCA2 and CCCTC binding factor (CTCF). Other NABPs involved in cellular processes and dysfunction include cellular nucleic acid-binding proteins (CNBP), which primarily bind to G-rich regions of nucleic acids, and Poly(A)-binding protein (PABP).

Among RBPs, mutations in TDP-43 and FUS are associated with ALS and FTD, while abnormalities in heterogeneous nuclear ribonucleoprotein A1 (hnRNP A1) are associated with a number of cancers, including lung and breast cancers. T-cell intracellular antigen 1 (TIA-1) is involved in stress granule formation and has been implicated in neurodegenerative diseases.

DBPs reported to contribute to cellular dysfunction include the tumor suppressor gene, p53, mutations of which are found in many cancers. Mutations in BRCA1 and BRCA2 genes are associated most frequently with hereditary breast and ovarian cancer and can increase the likelihood of a number of other cancers. CTCF is involved in chromatin organization and gene regulation and, when dysfunctional, appears to be linked to many cancers.

RBPs, a subset of NABPs, are ubiquitous regulatory proteins that bind to RNA through RNA-binding domains (RBD) and play an integral role in RNA metabolism and related molecular and cellular functions, including transcription, mRNA splicing, polyadenylation and, more generally, modification of RNA translation, transport, localization and turnover. They are made up of small RBDs and serve as key components of gene expression pathways in prokaryotic and eukaryotic cells.

Helicases, RBPs and other regulatory proteins can exist in multiple populations or isoforms within a cell which may or may not be spatially resolved to one or another physical compartment within the cell, so the nomenclature through which they are referenced can be misleading. In the case of the helicase G4R1, for example, nuclear and cytoplasmic isoforms are present both in the nucleus and in the cytoplasm and are transported between these two intracellular subcompartments, where the term subcompartment denotes a compartment within a cell. To avoid ambiguity, the term isoform as used herein refers to the biochemical pedigree of a target molecule without reference to its spatial localization within the cell at any given point in time. In some cases, e.g., with the helicase G4R1, the cytoplasmic isoform of an enzyme is likely pathogenic in the C9orf72 repeat expansion genetic background while the nuclear isoform is not. Thus, the cytoplasmic isoform of this enzyme is a viable therapeutic target (e.g., for ALS, FTD and Huntington's disease). Specificity for this isoform is an important drug discovery consideration to avoid compromising the function of the nuclear isoform, whose DNA and RNA resolvase roles are critical in DNA repair and gene regulation. The cytoplasmic isoform is key in regulating post-transcriptional processes, e.g., by preventing the accumulation of translationally inactive mRNAs with G4 structures in their untranslated regions. Dysregulation results in diseases such as ALS, a progressive neurodegenerative disease of motor neurons characterized by muscular wasting and spasticity. A repeat expansion mutation on the C9orf72 gene is the most common cause of various neurodegenerative diseases, including FTD and ALS. This gene mutation causes an abnormal excess of DNA and RNA structures called G-quadruplexes (G4s), which causes an accumulation of toxic G-quadruplex-knotted RNAs and through Repeat Associated Non-AUG Translation (RAN) toxic peptides to build up within a cell.

### SUMMARY OF THE INVENTION

The present invention relates to compositions and methods for modulating cell function at the isoform level of gene expression products and for treating genetic, degenerative, oncogenic and cellular diseases, including immune disorders, by selectively up- or downregulating at least one population of a cellular protein or therapeutic target. In one mode of operation, a novel composition of the invention comprises a cytoactive agent specifically crafted to target at least a first isoform of a target protein while lacking specificity for at least a second isoform of the target protein. The invention provides molecular constructs and methods that increase or decrease the activity of at least a first isoform of a target protein without substantially altering the activity of at least a second isoform of the target protein. In one mode of operation, the invention provides cytoactive agents that beneficially activate or inhibit a subpopulation of a cellular protein without perturbing another subpopulation in a manner that is therapeutic for the cell. In another mode of operation, isoforms, subpopulations and related variants of protein targets may reside in the cytoplasm, nucleus, mitochondrion, chloroplast or other organelle, location or compartment of the cell, optionally different organelles, locations or compartments of the same cell, of different cells in the same tissue or of different cells in different tissues. Therapeutic compositions are administered to humans, nonhuman animals or other biological species in a manner that is therapeutic for the organism. In another mode of operation, the invention provides kits to enable researchers and industrial scientists to selectively modulate either the nuclear or the cytoplasmic isoform of a protein of interest, e.g., a therapeutic target, preferentially over at least one other isoform of the target protein. In one embodiment, the therapeutic target is a NABP such as a helicase (e.g., G4R1 aka DHX36 and RHAU) or a member of the heterogeneous nuclear ribonucleoprotein family of proteins (e.g., TDP-43, FUS). In certain embodiments, the therapeutic target comprises a nucleotide localization signal (NLS), which can be used to therapeutic advantage as a means of preferentially targeting, for example, either the cytoplasmic or nuclear isoform of the target with minimal effect on other populations or isoforms. This ability to selectively modulate the activity of one population or isoform of a target protein over another is of particular interest in circumventing lethal or pathological consequences known to occur with the disruption of an essential protein, such as the nuclear isoform of G4R1.

G4R1 (more commonly referred to in the literature as DHX36 and RHAU) is a well-known G4 helicase enzyme initially reported by Vaughn et al. (JBC (2005) 280:38117-38120) that accounts for the majority of G4 helicase activity in human cells. G4R1 exists within distinct populations in a cell, residing in both the nucleus and cytoplasm. It has been shown that the presence of cytoplasmic G4R1 is likely to be therapeutically undesirable by enhancing production of toxic C9orf72 proteins via repeat-associated non-AUG translation (Smaldino et al., JBC 2021 August; 297(2):100914), which may exacerbate neurodegenerative diseases. In the C9orf72 repeat expansion carrier, the cytoplasmic G4R1 is essentially toxic and pathogenic and requires downregulation, while the nuclear isoform is an important regulatory, genome protective and signaling molecule that is to be preserved when targeting the cytoplasmic isoform for therapeutic purposes. In other words, nuclear G4R1 is nonpathogenic and therapeutically desirable, providing genomic stability and protecting against DNA damage. Thus, indiscriminate reduction of G4R1 may exacerbate FTD and/or ALS in patients with the C9orf72 repeat expansion mutation. It is also known that reduction of nuclear G4R1 activity disrupts cell cycle progression and cell division and plays a role in tumor progression.

G4R1 resides in a cell primarily as two isoforms, which present as distinct populations within each cell—a nuclear population and a cytoplasmic population. A first isoform (Isoform 1) contains a nuclear localization signal facilitating translocation of the protein into the nucleus (i.e., the nuclear population). The nuclear localization signal is not present in a second isoform (Isoform 2), which remains in the cytoplasm (i.e., the cytoplasmic population).

To selectively downregulate translation of Isoform 2, a therapeutic agent is configured to target the unique RNA sequence and/or novel protein structure that results from the splicing out of the nuclear localization signal (NLS) in the Isoform 2 mRNA, thus binding to either Spliceoform 2 or Isoform 2 and resulting in reduced translation of cytoplasmic G4R1 or downregulated effective cytoplasmic activity of Isoform 2 enzyme while maintaining native levels of nuclear G4R1 product. The therapeutic agent may comprise short interfering RNA (siRNA), short hairpin (shRNA), antisense oligonucleotide (ASO), or other molecules that directly target the cytoplasmic G4R1 Isoform 2 protein such as peptides, small molecules, antibodies, single chain antibodies, peptide mimetics and naturally occurring products selected for the ability to interact with the enzyme target or to lower the effective Isoform 2 helicase activity.

Although FTD and ALS are specifically mentioned in this disclosure, the methods described herein may be used to treat other repeat expansion disorders, including those in which the expanded repeat is capable of forming G4 structures on either or both strands or between strands such as but not limited to Huntington's disease, fragile X, fragile X tremor ataxia syndrome and other guanine-rich fragile sites, myotonic dystrophy type 2, spinocerebellar ataxia, cerebellar ataxia neuropathy, vestibular areflexia syndrome, myoclonic epilepsy, oculopharyngeal muscular dystrophy, and myotonic dystrophy type 1 as well as cancers and a host of other diseases for which there exist significant unmet clinical needs and for which selective targeting of nuclear or cytoplasmic targets is therapeutically desirable.

Compositions of the instant invention are of particular utility in modulating cellular function through isoform-specific protein interactions either in cell culture (e.g., for drug discovery purposes or for studying disease mechanisms), in isolated tissues and organs (e.g., for use in medical devices and regenerative medicine) and in whole organisms, as in human or veterinary therapeutics relying on isoform-selective targeting, NABPs represent an important class of molecules for modulating cellular activity or treating cellular disorders through isoform-specific interventions, because they are central actors in many cases of dysfunction at the cellular level. Prominent among NABPs implicated in cellular disorders and biological disease are the RBPs.

An object of the present invention is to provide a composition comprising a cytoactive agent for modulating repeat expansion products in a cell through subpopulation-specific interaction with a gene expression product of an endogenous wild-type nucleic acid-binding protein gene, said gene expression product having at least two subpopulations wherein the cytoactive agent modulates a first subpopulation of the at least two subpopulations and has minimal effect on a second subpopulation of the at least two subpopulations.

Another object of the present invention is to provide a preparation comprising a cytoactive agent formulated for use as one of a research reagent, a kit component and a therapeutic product for the prevention or treatment of a repeat expansion disorder.

Another object of the present invention is to provide a therapeutic composition comprising a subpopulation-specific cytoactive agent formulated for administration to a subject selected from the group consisting of a cell, a tissue, a culture, an organ, an implantable medical device and an organism.

Another object of the present invention is to provide a therapeutic composition comprising a subpopulation-specific cytoactive agent for administration to an organism wherein the organism is a human subject.

Another object of the present invention is to provide a method for extending the life of a subject, the subject comprising one of a cell, tissue, organ, implantable medical device and organism, by exposing the subject to a preparation incorporating a subpopulation-specific cytoactive agent of the invention.

Another object of the present invention is to provide a subpopulation-specific cytoactive agent wherein a first subpopulation of a targeted gene expression product comprises a first isoform of an endogenous wild-type NABP, and a second subpopulation of the targeted gene expression product comprises a second isoform of the endogenous wild-type NABP.

Another object of the present invention is to provide a subpopulation-specific cytoactive agent wherein the majority of a first subpopulation of an intracellular regulatory protein resides in a first cellular compartment, and the majority of a second subpopulation of the intracellular regulatory protein resides in at least a second cellular compartment.

Another object of the present invention is to provide a subpopulation-specific cytoactive agent wherein the first and second subpopulations of a targeted regulatory protein in a cell reside in the same cell or tissue.

Another object of the present invention is to provide a subpopulation-specific cytoactive agent wherein the first subpopulation of a targeted regulatory protein in a cell resides in a first cell or tissue, and the second subpopulation of the regulatory protein resides in a second cell or tissue.

Another object of the present invention is to provide a subpopulation-specific cytoactive agent with cellular compartment specificity wherein at least one cellular compartment is selected from the group consisting of centrosomes, chloroplasts, cytoplasm, cytoskeleton, endoplasmic reticulum, ER lumen, endosomes, Golgi apparatus, Golgi lumen, lysosomes, lysosomal lumen, mitochondria, mitochondrial matrix, inner membrane space, nucleus, nuclear envelope, nucleolus, peroxisomes, plasma membrane, ribosomes, vacuoles and membraneless organelles such as stress granules, Cajal bodies and P-bodies.

Another object of the present invention is to provide a subpopulation-specific cytoactive agent comprising an inhibitor of a first subpopulation of an endogenous gene expression product in a cell.

Another object of the present invention is to provide a subpopulation-specific cytoactive agent comprising an inhibitor of a first subpopulation of an endogenous gene expression product in a cell wherein the subpopulation-specific cytoactive agent is specific for a first isoform of the endogenous gene expression product in the cell and has minimal effect on a second isoform of the endogenous gene expression product in the cell.

Another object of the present invention is to provide a subpopulation-specific cytoactive agent comprising an agonist of a first subpopulation of an endogenous gene expression product in a cell.

Another object of the present invention is to provide a subpopulation-specific cytoactive agent comprising an agonist of a first subpopulation of an endogenous gene expression product in a cell that is specific for a first isoform of the endogenous gene expression product in a cell and has minimal effect on a second isoform of the endogenous gene expression product.

Another object of the present invention is to provide a subpopulation-specific cytoactive agent targeting an endogenous NABP in a cell wherein the endogenous NABP comprises at least one of a DNA-binding protein, an RNA-binding protein, a transcription factor, a helicase, a G4-helicase, a resolvase, G4R1 or a heterogeneous nuclear ribonucleoprotein.

Another object of the present invention is to provide a preparation comprising a subpopulation-specific cytoactive agent selected from the group consisting of low molecular weight ligands, small molecule inhibitors, RNAi, antisense oligonucleotides, ribozymes, gene editing constructs, proteins, antibodies, antibody mimics, nucleic acid aptamers, multivalent aptamers, peptides, peptide mimetics, natural product derivatives, mimetics and congeners, synthetic organic chemicals and compounds identified by combinatorial selection, in vitro molecular evolution, high-throughput screening and selection from diverse compound libraries.

Another object of the present invention is to provide a preparation comprising a subpopulation-specific cytoactive agent formulated for use as a therapeutic product for the prevention or treatment of a human disease comprising at least one of a cellular, immune, neurological, genetic, degenerative or oncogenic condition.

Another object of the present invention is to provide a preparation comprising a subpopulation-specific cytoactive agent formulated for use as a therapeutic product for prevention or treatment of a neurological condition comprising a neurodegenerative disease or a repeat expansion disorder.

Another object of the present invention is to provide a preparation comprising a subpopulation-specific cytoactive agent formulated as an inhibitor or an agonist of a disease-associated regulatory protein.

Another object of the present invention is to provide a preparation comprising a subpopulation-specific cytoactive agent targeting a regulatory protein that performs a plurality of functions wherein the cytoactive agent is a partial inhibitor or a partial agonist that affects only a subset of the plurality of functions.

Another object of the present invention is to provide a method for selectively modulating an endogenous NABP in a subject in a subpopulation-specific manner comprising administering a preparation of a subpopulation-specific cytoactive agent to the subject.

Another object of the present invention is to provide a method for selectively modulating an endogenous NABP in a subject in a subpopulation-specific manner comprising administering to the subject a preparation of a subpopulation-specific cytoactive agent comprising an oligonucleotide selected from the group consisting of RNA, DNA, RNAi, shRNA, siRNA, a nucleic acid aptamer, a multivalent aptamer, an antisense oligonucleotide, a ribozyme, a conjugated or immobilized nucleic acid molecule and an oligonucleotide mimetic comprising nonnaturally occurring nucleotides, abasic nucleotides or backbone modifications.

Another object of the present invention is to provide a method for selectively modulating an endogenous NABP in a subject in a subpopulation-specific manner comprising administering to the subject a preparation of a subpopulation-specific cytoactive agent comprising an RNAi expression plasmid delivered via an attenuated virus.

Another object of the present invention is to provide a method comprising administering to a subject a preparation of a subpopulation-specific cytoactive agent comprising an RNAi expression plasmid delivered via an attenuated virus wherein the preparation is formulated for administration to a human subject.

Another object of the present invention is to provide a kit for the subpopulation-specific manipulation of an intracellular regulatory protein existing in a plurality of subpopulations within cells wherein the kit comprises i) a pool of synthetic heteropolymers comprising candidate molecules that specifically interact with a first subpopulation of the intracellular regulatory protein and do not specifically interact with a second subpopulation of the intracellular regulatory protein; ii) a transfection reagent for delivering the synthetic heteropolymers to the cells; and iii) a fractionating reagent for separating the first and second subpopulations of the intracellular regulatory protein.

Another object of the present invention is to provide a kit for the subpopulation-specific manipulation of an intracellular regulatory protein wherein synthetic heteropolymers of the kit comprise candidates selected from the group consisting of peptides, peptide mimetics, antibodies, antibody fragments, antibody mimetics, RNAi, antisense oligonucleotides, aptamers, multivalent aptamers, ribozymes, oligonucleotide conjugates, immobilized oligonucleotides and mimetics comprising nucleotide congeners, abasic nucleotides and backbone modifications.

Another object of the present invention is to provide a kit for the subpopulation-specific manipulation of an intracellular regulatory protein which further comprises subpopulation-specific marker antibodies to confirm successful fractionation of a first and a second subpopulation of the intracellular regulatory protein.

Another object of the present invention is to provide a kit for the subpopulation-specific manipulation of an intracellular regulatory protein wherein a first and a second subpopulation of the intracellular regulatory protein comprise different isoforms of the intracellular regulatory protein.

Another object of the present invention is to provide a kit of for the subpopulation-specific manipulation of an intracellular regulatory protein wherein a majority of a first subpopulation of the intracellular regulatory protein resides in a first cellular compartment, and a majority of a second subpopulation of the intracellular regulatory protein resides in a second cellular compartment.

Another object of the present invention is to provide a kit for the subpopulation-specific manipulation of an intracellular regulatory protein wherein a majority of a first subpopulation of the intracellular regulatory protein and a majority of a second subpopulation of the intracellular regulatory protein reside in the same cell or tissue.

Another object of the present invention is to provide a kit for the subpopulation-specific manipulation of an intracellular regulatory protein wherein a majority of a first subpopulation of the intracellular regulatory protein resides in a first cell or tissue, and a majority of a second subpopulation of the intracellular regulatory protein resides in a second cell or tissue.

Another object of the present invention is to provide a research reagent comprising a component of a kit for the subpopulation-specific manipulation of an intracellular regulatory protein.

Another object of the present invention is to provide a composition for modulating biological function, said composition comprising a subpopulation-specific cytoactive agent configured to bind an mRNA sequence coding for a cytoplasmic NABP lacking a nuclear localization signal.

Another object of the present invention is to provide a composition for modulating biological function, said composition comprising a subpopulation-specific cytoactive agent configured to bind an mRNA sequence coding for a cytoplasmic NABP lacking a nuclear localization signal wherein the cytoplasmic NABP comprises at least one of a DNA-binding protein, an RNA-binding protein, a transcription factor, a helicase, a G4-helicase, a resolvase, G4R1 or a heterogeneous nuclear ribonucleoprotein.

Another object of the present invention is to provide a method for treating a subject with a disease-associated NABP comprising administering to the subject a preparation comprising a composition for modulating biological function, said composition comprising a subpopulation-specific cytoactive agent configured to bind an mRNA sequence coding for a cytoplasmic NABP lacking a nuclear localization signal.

Another object of the present invention is to provide a method for treating a subject with a disease-associated NABP comprising administering to the subject a preparation comprising at least one of a DNA-binding protein, an RNA-binding protein, a transcription factor, a helicase, a G4-helicase, a resolvase, G4R1 or a heterogeneous nuclear ribonucleoprotein.

Another object of the present invention is to provide a method of inhibiting at least one of the formation, aggregation or accumulation of a repeat expansion product in a cell comprising a plurality of subpopulations of an endogenous NABP, the method comprising i) detecting the presence of the repeat expansion product in the cell, ii) identifying the endogenous NABP causally associated with the formation, aggregation or accumulation of the repeat expansion product, and iii) administering to the cell a cytoactive agent that modulates at least one but less than all of the plurality of subpopulations of the endogenous NABP in a manner that alters the amount, activity, rate of formation, rate of degradation or rate of accumulation of the repeat expansion product in the cell.

Another object of the present invention is to provide a method of modulating biological function by inhibiting the formation, aggregation or accumulation of a repeat expansion product in a cell wherein the method comprises administering to the cell a cytoactive agent formulated as a therapeutic preparation for the prevention or treatment of a repeat expansion disorder.

Another object of the present invention is to provide a method of modulating biological function by inhibiting the formation, aggregation or accumulation of a repeat expansion product in a cell wherein the therapeutic preparation is formulated as a biopharmaceutical product for administration to a human subject.

Another object of the present invention is to provide a preparation of a cytoactive agent formulated as a therapeutic product comprising an endogenous NABP modulator selected from the group consisting of low molecular weight ligands, small molecule inhibitors, RNAi, antisense oligonucleotides, ribozymes, gene editing constructs, proteins, antibodies, antibody mimics, nucleic acid aptamers, multivalent aptamers, peptides, peptide mimetics, natural product derivatives, mimetics and congeners, synthetic organic chemicals and compounds identified by combinatorial selection, in vitro molecular evolution, high-throughput screening or selection from diverse compound libraries.

It will be appreciated that the various methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 illustrates a method and composition for selectively downregulating a cytoplasmic population of G4R1 using shRNA.

FIG. 2 illustrates isoform-specific modulation of a target NABP based on isoform specificity of a therapeutic molecule designed to modulate one isoform of the NABP in a first cellular compartment and not another isoform of the same NABP that resides in a second cellular compartment.

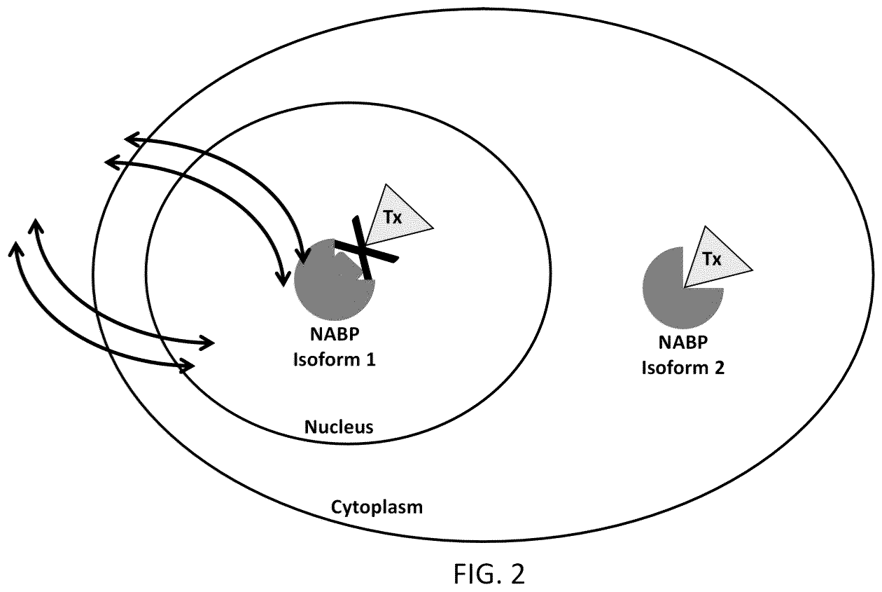

FIG. 3 illustrates isoform-specific modulation of a target NABP based on isoform specificity of a therapeutic molecule designed to modulate one isoform of the NABP in a cellular compartment and not another isoform of the same NABP that resides in the same cellular compartment.

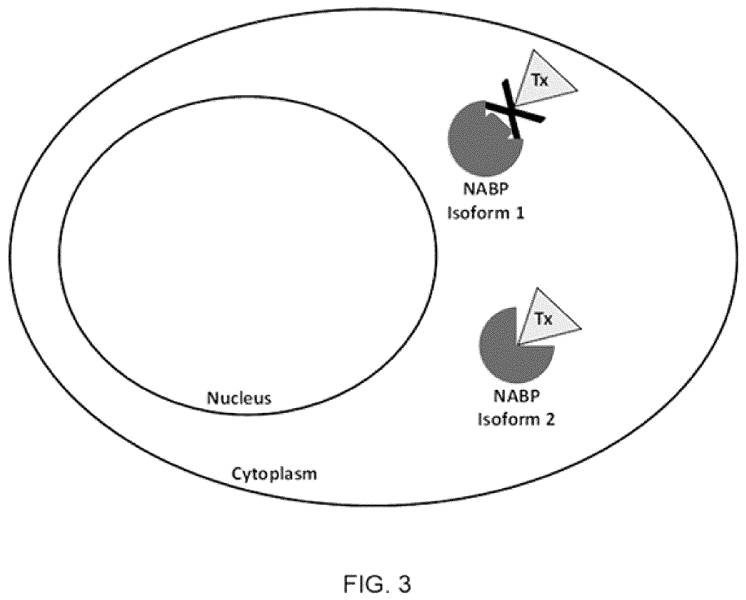

FIG. 4 illustrates cell compartment-specific modulation of a target NABP based on cell compartment exclusion of a therapeutic molecule designed to cross into the cytoplasm and be excluded from nucleus.

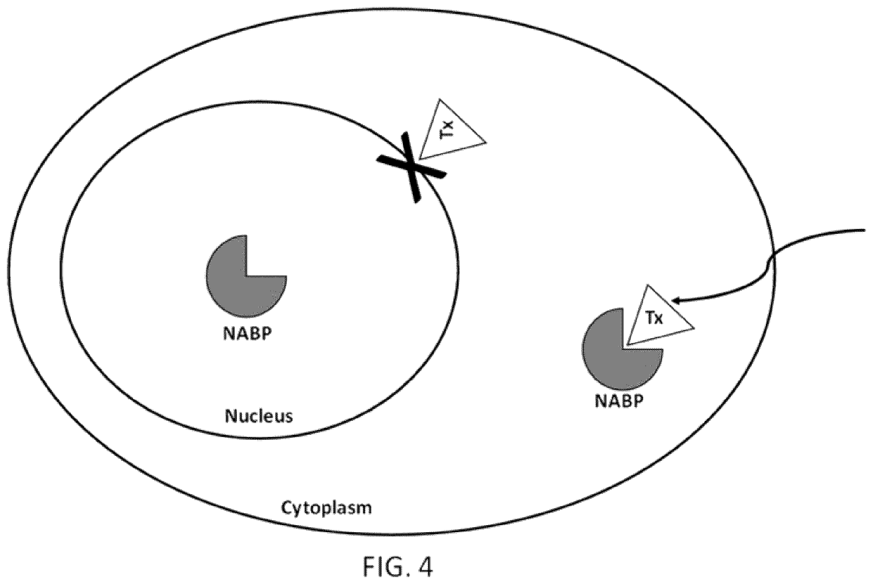

FIG. 5 illustrates the targeting of a NABP based on isoforms or subpopulations of a NABP that resides in separate cells or tissues. The illustration shows modulation of a NABP by a therapeutic molecule (Tx) in one cell or tissue and not in a second cell or tissue where the NABP also resides.

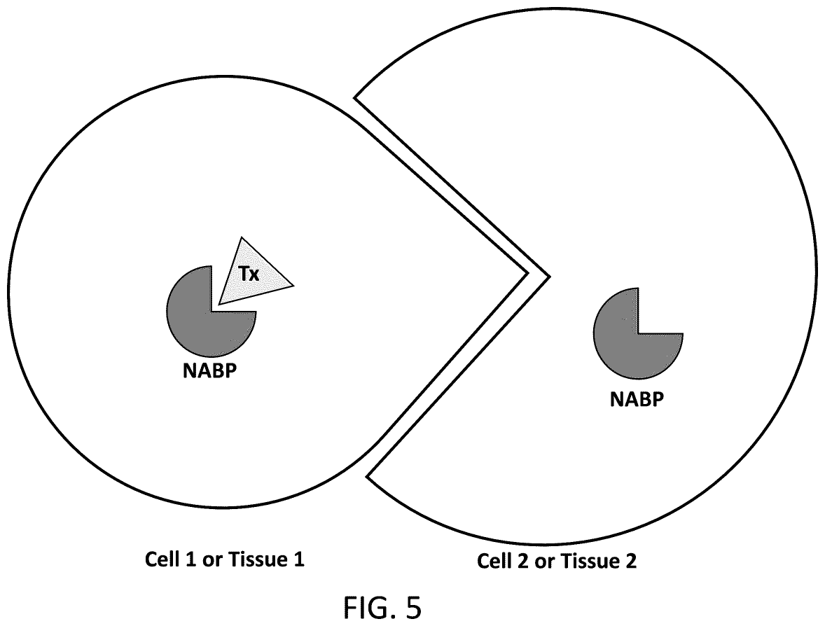

FIG. 6 illustrates the targeting of a specific function of a NABP among a plurality of functions of the NABP. The illustration shows therapeutic molecule (Tx) modulation of the G-quadruplex binding domain (DSM) of an exemplary NABP, G4R1, while leaving the AU-rich mRNA decay function (AU) of G4R1 unmodulated.

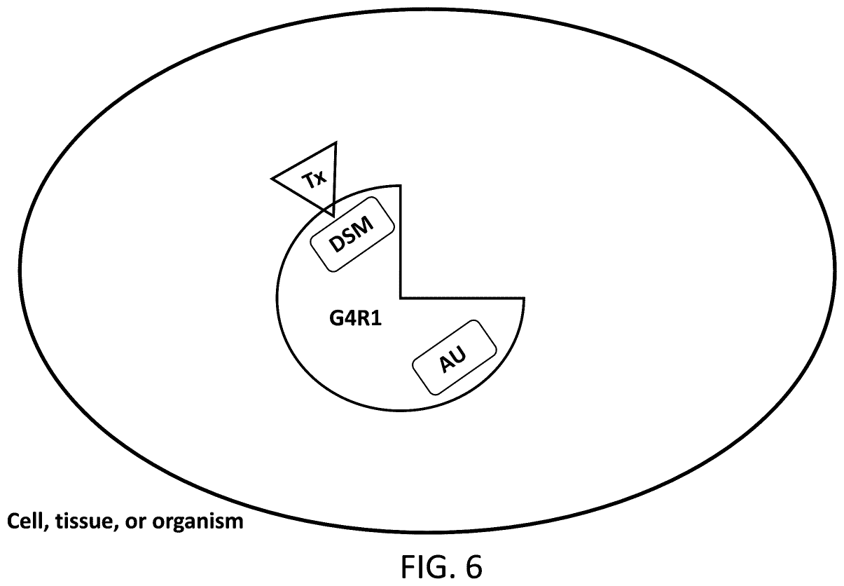

FIG. 7 illustrates an example of an isoform-specific experimental strategy to modulate G4R1. (A) Human mRNA sequences of G4R1 variant 1 containing a nuclear localization signal (NLS) sequence (top) and variant 2 with NLS removed (bottom). ASO's shown staggered across NLS splice junction site unique to variant 2. (B) ASO sequences targeted to G4R1 variant 2. (C) G4R1 NLS sequence (shaded) is well conserved across humans, mice, flies, worms, and yeast (Tran et al.) rendering the above strategy in humans applicable to other species. This strategy is highly modifiable by for example, including a number of different fixed size target-effective oligomer sets located around the splice NLS junction site.

### DETAILED DESCRIPTION OF THE PREFERRED EMBODIMENTS

### Abbreviations

Å: Angstrom, a unit of length equal to one hundred-millionth of a centimeter, (10⁻¹⁰ meter)
ADME: Absorption, distribution, metabolism and excretion
AFM: Atomic force microscopy
ALS: Amyotrophic lateral sclerosis
ARE: AU-rich element
ASO: Antisense oligonucleotide
ATP: Adenosine triphosphate
BRCA1: Breast cancer type 1 susceptibility protein
BRCA2: Breast cancer type 2 susceptibility protein
BSE: Bovine spongiform encephalopathy
CNBP: Cellular nucleic acid-binding protein
CTCF: CCCTC-binding factor
CWD: Chronic wasting disease
DAA: D-amino acid
DBP: DNA-binding protein
DDX5: A DEAD-box helicase
DEAD-box: Asp-Glu-Ala-Asp (amino acid sequence)
DEAH/RHA helicase: Enzyme that remodels ribonucleoprotein (RNP) complexes in central processes of RNA metabolism such as transcription, splicing, or translation
DHX36: A eukaryotic DEAH/RHA family helicase that disrupts G-quadruplex structures (G4s) with high specificity and, as used herein, refers to either the helicase gene, transcript or gene product corresponding to the enzyme G4 Resolvase 1 (G4R1) initially reported by Vaughn et al. (JBC (2005) 280:38117-38120)
DEXH: A protein family essential for RNA metabolism, DNA replication and the replication of many viruses
DM2: Myotonic dystrophy type 2
DNA: Deoxyribonucleic acid
DOTMA: N-[1-(2,3-dioleyloxy)propyl]-N,N,N-trimethylammonium chloride, a cationic lipid commonly used in gene therapy as a non-viral vector to deliver genetic material into cells
DSM: DHX36-specific motif of the G4-helicase G4R1
ER: Endoplasmic reticulum
FeLV: Feline leukemia virus
FITC: Fluorescein isothiocyanate
FRET: Fluorescence resonance energy transfer
FTD: Frontotemporal dementia
FUS: Fused in sarcoma protein
FUS/TLS: Fused in sarcoma/translocated in liposarcoma, an RBP involved in RNA metabolism, protein translation and cellular transport
G4R1: G4 Resolvase 1
G4: G-quadruplex
GFP: Green fluorescent protein
hnRNP A1: Heterogeneous nuclear ribonucleoprotein A1
HTS: High-throughput screening
HuB/ELAVL2: An RBP involved in oogenesis and implicated in autism spectrum disorder
HuR: A Hu protein
IC₅₀: Concentration required for 50% inhibition
Kd: Dissociation constant
K-Res: A buffer for determination of helicase activity
LLM: Large language model
MCM: Minichromosome maintenance
mRNA: Messenger RNA
NABP: Nucleic acid-binding protein
NLS: Nuclear localization signal
nt: Nucleotide
p53: Tumor protein p53
PABP: Poly(A)-binding protein
PCR: Polymerase chain reaction
PEG: Polyethylene glycol
QKI: Quaking protein
RAN: Repeat-associated non-AUG (as in translation)
RBP: RNA-binding protein
RBD: RNA-binding domain
RED: Repeat expansion disorder
RHAU: RNA helicase associated with AU-rich RNA
RIPA: Radioimmunoprecipitation buffer
RNA: Ribonucleic acid
RNAi: RNA interference
RNP: Ribonucleoprotein
RSM: RHAU-specific motif of the G4-helicase G4R1
SELEX: Systemic Evolution of Ligands by EXponential enrichment
SFM: Scanning force microscopy
shRNA: Short hairpin RNA
siRNA: Short interfering RNA
TAMRA: 5-Carboxytetramethylrhodamine
TBE: A nucleic acid buffer most commonly used in electrophoresis
TDP-43: TAR DNA-binding protein 43
TIA-1: TIA1 cytotoxic granule-associated RBP
TIAR: T-cell intracellular antigen 1-related/like, an RBP that regulates gene expression and is involved in cell death
TR: Thyroid hormone receptor
UTR: Untranslated region
WRN: Werner syndrome

The details of one or more embodiments of the presently-disclosed subject matter are set forth in this document. Modifications to embodiments described in this document and other embodiments will be evident to those of ordinary skill in the art after a study of the information provided in this document. The information provided in this document, and particularly the specific details of the described exemplary embodiments, is provided primarily for clearness of understanding, and no unnecessary limitations are to be understood therefrom. In case of conflict, the specification of this document, including definitions, will control.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount is meant to encompass variations of ±10% of the most precise digit in the value or amount (e.g., “about 1” refers to 0.9 to 1.1, “about 1.1” refers to 1.09 to 1.11, etc.).

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Remediation refers to methods and accompanying compositions for achieving and maintaining cellular homeostasis. This encompasses not only therapeutic purposes, but also, more broadly, the tuning and alignment of intracellular regulatory networks. To this end, kits, research reagents and methods described herein for subpopulation-specific modulation and manipulation of target molecules—including the high-resolution isolation and purification of expressed gene products—are provided. The overarching purpose of these offerings is to achieve homeostatic alignment through the discovery and resolution of pathogenic mechanisms.

### Definitions of Selected Terms

“Nonnaturally occurring” refers to a composition which is synthetic, has been altered or modified from its natural state, or otherwise not heretofore found in nature.

“Cytoactive agent” is a composition of matter having activity toward a cell.

“Modulating” means transforming, increasing or decreasing the amount, activity or function of a target, including relocating the target within a cell, and is typically used in reference to a designed or determined desirable effect of a cytoactive agent, preparation or therapeutic composition on an intracellular molecular target, signaling pathway or regulatory network.

“Regulatory network” refers to one of several intracellular stimulus-response ecosystems comprising NABPs that collaborate in maintaining cellular and biological homeostasis in response to internal and external environmental conditions and challenges. A mutation on the C9orf72 gene is a common cause of a disruption to a regulatory network which results in the various neurodegenerative diseases described herein as indications worthy of treatment using cytoactive agents, preparations and therapeutic compositions of the instant invention.

“Subpopulation-specific” refers to the target-directed selectivity and preferential modulation of spatiochemically discernable isoforms and compartmentalized localizations of expressed gene products, particularly intracellular NABPs and associated RNA transcripts of the instant invention.

“Gene expression product” refers to both transcription and translation products of genes, including but not limited to, genes coding for NABPs, transcribed into mRNA with or without nuclear localization signals that mediate transport between intracellular (cytoplasmic vs. nuclear) compartments.

“Wild-type” refers to the naturally occurring target molecules encoded and expressed through the native genome, transcriptome and proteome of plant, nonhuman animal and human subjects and beneficiaries of the invention and not to mutated genes or gene products targeted by therapeutic products designed to address or overcome a genetic mutation.

“Catalytic,” when used in connection with nucleic acid-binding proteins, refers to nucleic acid-binding proteins having enzymatic activity toward their molecular and cellular substrates.

“Nucleic acid-binding protein gene” refers to the gene and gene products encoded by NABP molecular machinery including the RNA transcripts and protein products of NABP genes.

“Gene expression product” refers to the RNA and protein products expressed by transcription and translation of a particular gene.

“Subpopulations” are structurally, biochemically, functionally or spatially distinct subsets of a target molecule, typically a gene expression product, that exist in different isoforms or intracellular compartments.

“Preparation” means a composition prepared for a particular application, e.g., use as a research reagent, kit, kit component or therapeutic product for the treatment of a subject or population.

“Research reagent” means a chemical, biochemical or biological preparation for use in laboratory-based research.

“Kit” refers to a commercial or home-brewed product for performing a method of the invention and includes kit components, including, in some embodiments, a package insert or user's manual.

“Kit component” means a research reagent, ingredient, supply, device or package insert used in combination with at least one other such constituent to accomplish a defined purpose (the “intended use”).

“Therapeutic product” refers to a cytoactive agent, preparation or therapeutic composition formulated for administration to a subject and/or prevention or treatment of a disease, disorder or condition.

“Subject” means a recipient of a cytoactive agent, preparation, therapeutic product or therapeutic composition and includes a cell, group of cells, tissue, organ, organism or population advantageously comprising a plant, nonhuman animal or human.

“Cell-based composition” means an experimentally, preventively or therapeutically modified cell or plurality of cells comprising a cytoactive agent, preparation, therapeutic product or therapeutic composition.

Pharmaceutically acceptable carriers (aka pharmaceutical carriers) are vehicles for delivering a therapeutic composition of the invention to a patient and include agents attached to or formulated with the active ingredient of the therapeutic composition for the purpose of optimizing pharmacokinetic properties, e.g., the absorption, distribution, metabolism and/or excretion (ADME) properties of the active ingredient. Examples of pharmaceutical carriers include, for example and without limitation, solubilizing agents, peptides, proteins, polymers, microparticles, nanostructures, liposomes, micelles, emulsifiers and the like. Pharmaceutical carriers are important for improved delivery, as many drugs are poorly soluble in water or have difficulty penetrating cell membranes on their own. Carriers can help overcome these limitations by encapsulating the drug or altering its properties, allowing for better absorption and delivery to the target site. Pharmaceutical carriers can be designed to release the drug slowly over time, reducing the need for frequent dosing and potentially minimizing side effects. Some carriers can be engineered to target specific tissues or organs, minimizing exposure to healthy cells and improving the drug's effectiveness. Examples of commonly used pharmaceutical carriers include liposomes, polymeric micelles, nanoparticles, cyclodextrins and polyethylene glycol (PEG), among many others. Liposomes are microscopic spheres made of phospholipids and other molecules with fatty acid chains and quaternary ammonium head groups such as DOTMA, adherent to components of cell membranes. They can encapsulate drugs and deliver them directly to cells. Polymeric micelles are microstructures formed from amphiphilic polymers with a hydrophobic core and a hydrophilic shell that can solubilize hydrophobic drugs and deliver them to specific sites. Cyclodextrins are cyclic sugar molecules that can form inclusion complexes with drugs, improving their solubility and stability. Nanoparticles are delivery systems on the nanometer scale made from various materials like polymers, lipids and metals which are useful for targeted delivery and controlled release. PEG is a stable, water-soluble polymer with low toxicity used as an excipient in many pharmaceutical products in oral, topical, and parenteral dosage forms to improve solubility, bioavailability and other pharmacokinetic properties. The choice of pharmaceutical carrier for a particular drug depends on various factors, including the drug's properties, desired delivery method and target site. In some embodiments, the pharmaceutical carrier comprises a permeabilizing agent.

Permeabilizing agents are substances that temporarily increase the permeability of cell membranes, allowing larger molecules like peptides, proteins and oligonucleotides to enter cells. They may be attached to or formulated with a therapeutic composition of the invention or they may be administered independently, whether simultaneously or sequentially. Attention to permeabilizing agents is crucial for delivering therapeutic agents that target intracellular targets, as these molecules typically cannot passively cross the cell membrane. Examples of permeabilizing agents include, without limitation, cell-penetrating peptides, liposomes, viral vectors, amphiphiles, lipopeptides, dendrimers and the like, or they may be permeabilization methods such as electroporation or sonoporation. Factors to consider when choosing a permeabilizing agent for a particular indication include, e.g., 1) target cell type, as different cell types have different membrane properties; 2) therapeutic agent properties, as the size, charge and hydrophobicity of the therapeutic agent will influence its ability to enter cells with the help of a permeabilizing agent, 3) toxicity, as permeabilizing agents can be toxic to cells, and 4) delivery efficiency, which will depend on the choice of permeabilizing agent and the formulation of the therapeutic agent. Challenges in the use of permeabilizing agents include, without limitation, 1) non-specific delivery, e.g., effects on non-target cells, leading to off-target effects, 2) toxicity, as some permeabilizing agents are toxic to cells, especially at high concentrations, 3) stability, as therapeutic agents may be degraded or inactivated by the permeabilizing agent or the intracellular environment.

Efforts to develop permeabilizing agents for delivering synthetic nucleic acids like ASOs and aptamers into cells in vivo have advanced rapidly in recent years. Researchers have been working on designing novel cationic and procationic amphiphiles that can efficiently pack, transport and deliver nucleic acids to various targets in the body. Recent developments include self-assembled synthetic gene delivery systems, which are gaining attention for their potential to overcome delivery barriers. Though safer than viral vectors, these systems still lag behind in terms of efficiency. Structure-activity relationships have been established for different amphiphile classes, facilitating design of improved in vivo nucleic acid delivery. Cationic and procationic amphiphiles are being explored for their ability to form stable complexes with nucleic acids, facilitating efficient delivery. Dendrimers and lipopeptides are also being investigated for their potential as permeabilizing agents. Surface-active agents are being designed to improve the stability and efficacy of nucleic acid delivery systems. For neurological therapeutics of the instant invention, intrathecal injection remains the delivery system of choice, though advances described herein promise to expand delivery options, which must be tuned to the requirements of a given combination of target and therapeutic construct.

Membrane delivery systems are compositions and associated methods for delivering drugs with high efficiency to specific sites in the body, advantageously at a controlled rate or time. These delivery systems can be used for a variety of applications, including oral administration, implants, patches and transdermal drug delivery. Membrane delivery systems include, without limitation, osmotic membrane systems, diffusion-controlled membrane systems and the outer cell membrane itself, advantageously modified by permeabilizing agents to enable the membrane to act as a form of enhanced delivery system by protecting drugs comprising pharmaceutical carriers (e.g., nanoparticles, proteins, antibodies or functionalized liposomes) from degradation or immune surveillance.

Neuroprotective agents are substances that target various aspects of disease processes, including oxidative stress, inflammation and cell death. Examples include, but are not limited to, free radical scavengers and antioxidants such as vitamin E and coenzyme Q10, which may protect neurons from oxidative stress damage, as well as anti-inflammatory drugs, which may help to reduce inflammation in the nervous system.

Nucleotides, aka oligonucleotides, are compositions comprising nucleoside bases and include, without limitation, nucleotide bases, nucleic acids and oligonucleotides of natural and synthetic origin, including constructs comprising nucleotide analogs, derivatives, conjugates and backbone modifications.

Nucleotide analogs are molecules that can be used in place of naturally occurring bases in nucleic acid synthesis and processing, optionally enzymatic as well as chemical synthesis and processing. They include modified nucleotides capable of base pairing and synthetic bases other than adenine, guanine, cytosine, thymidine, uracil and minor bases. Examples include, without limitation, modified purines and pyrimidines, minor bases, convertible nucleosides, structural analogs of purines and pyrimidines, labeled, derivatized and modified nucleosides and nucleotides, conjugated nucleosides and nucleotides, sequence modifiers, terminus modifiers, spacer modifiers and nucleotides with backbone modifications, e.g., ribose-modified nucleotides, phosphorothioates, phosphonamidites, phosphoramidates, methyl phosphonates, methyl phosphoramidites, methyl phosphonamidites, 5′-.beta.-cyanoethyl phosphoramidites, peptide nucleic acids, methylenephosphonates, phosphorodithioates, achiral and neutral internucleotidic linkages and nonnucleotide bridges like polyethylene glycol, aromatic polyamides and lipids.

A population of molecules within a cell is a set of molecules that share a common structure, activity or cellular localization. Targeted populations of molecules, also referred to as subpopulations, include subsets or isoforms of a given population that are either biochemically or spatially defined subsets of the population that are amenable to selective modulation in accordance with methods disclosed herein. Helicases, for example, may be distributed between different isoforms, e.g., cytoplasmic vs. nuclear isoforms, or different cellular compartments, e.g., the cytoplasm vs. the nucleus. The helicase, G4R1, for example, exists in different isoforms (aka subpopulations), i.e., isoforms 1 and 2, also referred to as the nuclear and cytoplasmic isoforms, respectively, which may or may not be spatially segregated into different cellular compartments. In other words, both the cytoplasmic and nuclear isoforms of G4R1 may reside in either the cytoplasm or the nucleus and distribute between these two compartments. Importantly, the cytoplasmic isoform of G4R1 can generate through RAN translation toxic peptides implicated in a number of neurological diseases (e.g., ALS, FTD, Huntington's disease and Fragile X syndrome). Unlike the nuclear isoform of G4R1, which provides genomic stability and protects against DNA damage, the cytoplasmic isoform in the C9orf72 expanded repeat genetic context is considered to be a pathogenic protein and potentially attractive therapeutic target.

Fractionation of cellular populations of molecules refers to the discernment and/or physical separation of constituent subpopulations in accordance with either their biochemical properties or spatial localization within the cell.

Repeat expansion disorders (RED) are a group of inherited neurological and neuromuscular diseases caused by an expansion of DNA repeats.

Compositions of the instant invention that exert their effects through actions on or in a ceil may be referred to herein as actives, active ingredients or cytoactive agents.

This invention turns on leveraging the unique structures and properties of different isoforms of a given protein to target at least one subpopulation of the protein for the therapeutic benefits of improved specificity, reduced toxicity and/or improved efficacy. While many strategies disclosed herein leverage the specificity benefits of preferentially up- or downregulating either a cytoplasmic or nuclear isoform of a target protein, a vast array of therapeutic mechanisms can be devised through attention to the combination of molecular effect on the target isoform (e.g., activation vs. repression, stimulation vs. inhibition, upregulation vs. downregulation), cellular localization or prevalence (e.g., nucleus vs. cytoplasm, mitochondrion, chloroplast, membrane, cell wall or other cellular organelle), alternative splicing dynamics and the realm of isoform diversity among different cellular proteins and therapeutic targets, particularly NABPs comprising different isoforms that can serve as handles for enhanced specificity of therapeutic agents such as nucleotide, peptide, protein, antibody, small molecule, modified natural product and biomimetic ligands, targeting agents and molecular editing constructs.

Protein isoform diversity arises from alternative splicing, post-translational modification and alternative promoter usage, generating multiple forms of the same protein with distinct structures, functions and subcellular localizations. This diversity is particularly notable in NABPs like hnRNPs, RBPs, helicases and other essential regulators of gene expression. By producing isoforms with different properties, cells achieve fine control over RNA processing, transport, translation and stability. This isoform-specific targeting capability is of substantial interest for research and therapeutic applications, where precision modulation of specific isoforms enables tailored cellular responses or mitigation of disease states driven by misregulated or pathogenic protein variants.

RBPs demonstrate extensive isoform diversity. For example, hnRNP A1, a multifunctional protein involved in RNA splicing, transport, and translation, exists in numerous isoforms with distinct RNA-binding affinities and cellular localizations. Isoforms of hnRNP A1 contribute to a variety of RNA processing activities by modulating exon inclusion or exclusion, enabling context-specific gene regulation. Tropomyosin, another highly spliced protein, plays a central role in muscle contraction and cell motility, producing isoforms that meet the functional requirements of different tissue types. Calmodulin, known for its role in calcium-mediated signaling, undergoes various post-translational modifications, yielding isoforms with unique regulatory properties that adjust cellular responses to changing calcium levels.

Protein isoform diversity is also present in proteins with specific cellular or stress-related functions. For instance, Hu proteins, such as HuR, modulate mRNA stability in response to stress, with their isoforms impacting cell proliferation and stress responses through distinct RNA-binding preferences. Similarly, the Quaking protein (QKI) family, important in RNA splicing and localization, has isoforms that facilitate neuronal differentiation and myelination. In stress conditions, TIA-1 and TIAR isoforms promote stress granule formation, helping cells to transiently inhibit translation and conserve resources.

Isoform diversity extends to helicases, essential enzymes for nucleic acid unwinding. For example, DDX5, a DEAD-box helicase, has isoforms involved in RNA processing events, while RecQ helicase isoforms contribute to DNA repair and genomic stability. These isoform variations in structure and activity are central to cellular adaptation in response to replication stress or DNA damage.

Understanding isoform diversity allows for the development of isoform-specific therapeutic strategies, enhancing the precision of interventions targeting disease-relevant protein functions without affecting other isoforms that may have essential physiological roles. Such approaches hold promise in tackling disorders characterized by aberrant splicing, seemingly random post-translational modifications or other dysregulated isoform-specific activities, paving the way for targeted molecular and cell biology research applications. Considerations of the isoform diversity of NABPs requires attention to cellular localization and transport.

### Diversity of NABP Isoforms

NABPs are a diverse group of proteins that interact with DNA and RNA, playing essential roles in a wide range of cellular processes. These proteins exhibit remarkable isoform diversity generated through various mechanisms, including alternative splicing, post-translational modifications and differential gene expression. This diversity allows NABPs to perform a wide range of functions in different cellular contexts.

### Isoform Diversity and Cellular Localization

NABPs are found in various cellular compartments, each with its specific function. In the nucleus, DNA-binding proteins interact directly with DNA and regulate crucial cellular processes such as transcription, replication, DNA repair and chromatin remodeling. RBPs in the nucleus play pivotal roles in RNA processing, transport, stability and translation.

In the cytoplasm, many RBPs function to regulate translation, mRNA stability and mRNA localization. Additionally, certain organelles, such as mitochondria and chloroplasts, possess their own DNA and RNA. These organelles harbor specific NABPs essential for mitochondrial and chloroplast gene expression and energy metabolism. The specific localization of a NABP can vary depending on its function, cell type and cellular state. Some proteins may shuttle between different compartments, while others are permanently localized to a specific location.

Notable Examples of NABPs with Isoform Diversity

The heterogeneous nuclear ribonucleoprotein (hnRNP) family provides a clear example of isoform diversity. For instance, hnRNP A1 has multiple isoforms differing in RNA-binding domains and nuclear localization signals, each playing diverse roles in RNA processing. hnRNP C contains several isoforms that regulate alternative splicing and mRNA stability, while hnRNP D includes multiple isoforms involved in RNA processing and transcriptional regulation.

The Hu protein family also illustrates the impact of isoform diversity. HuR (ELAVL1) has multiple isoforms that regulate mRNA stability and translation, influencing cell proliferation, differentiation and stress responses. HuB (ELAVL2), on the other hand, has isoforms involved in neuronal development and function.

The Quaking protein (QKI) with its three major isoforms (QKI-5, QKI-6, and QKI-7) demonstrates how isoform diversity can impact functions such as alternative splicing and mRNA localization, with each isoform exhibiting different expression patterns. TIA-1 and TIAR are other examples of proteins with isoforms that regulate mRNA stability and translation during cellular stress responses. The FUS/TLS protein has multiple isoforms involved in RNA processing, transcription, and DNA repair.

Helicases, such as DEAD-box helicases and RecQ helicases, show notable isoform diversity. DEAD-box helicases, exemplified by DDX5, participate in various RNA processing events, while RecQ helicases, such as RECQ1, function in different cellular compartments and respond to diverse cellular stresses. Other helicases, such as MCM helicases, form complexes of six proteins, each with multiple isoforms essential for DNA replication. The Werner syndrome helicase (WRN) is also notable for its isoform diversity, which supports roles in DNA replication, repair and telomere maintenance.

The diversity of NABP isoforms contributes to the complexity of gene expression regulation and cellular function. By understanding the specific functions of different NABP isoforms, researchers can gain insights into the molecular mechanisms underlying various cellular processes and human diseases.

### EXAMPLES

Following are examples intended to illustrate selected embodiments of the invention through methods that are readily adaptable to experimental protocols routinely practiced in modern biochemistry, molecular biology, cell biology and clinical laboratories. These examples are illustrative rather than exhaustive, nonlimiting and provided purely for explanatory purposes. They are intended to highlight a few embodiments of subpopulation-directed remediation in ways that encompass methods and compositions designed to achieve and maintain cellular homeostasis, including tuning and alignment of intracellular regulatory networks, high-resolution modulation, isolation and purification of expressed gene products and accompanying therapeutic strategies designed to resolve pathogenic mechanisms.

### Example 1: General Strategies for Therapeutic Targeting of RBPs as Illustrated by Helicase-Directed Drug Discovery

RNA-binding proteins are, as the name suggests, proteins that bind to RNA molecules. They play a vital role in various cellular processes, including RNA processing, stability, transport, translation, localization and overall function, e.g.:
1. splicing, capping, and polyadenylation of RNA molecules,
2. regulating the localization and half-life of RNA molecules and determining how long they remain active,
3. facilitating the transport of RNA molecules from the nucleus to the cytoplasm,
4. influencing the efficiency of protein synthesis from mRNA and
5. guiding RNA molecules to specific cellular locations.

As such, RBPs are key regulators of gene expression, influencing which genes are expressed and at what levels. They are involved in a wide range of biological processes, from cell growth and differentiation to disease development, including diseases for which the instant invention offers solutions, particularly genetic, cellular, degenerative and oncogenic diseases and the methods, tools and kits for applying the invention to target-directed compositions, methods, research kits and constituent reagents for biomedical applications, e.g., therapeutics, drug discovery and research products.

Transport between nucleus and cytoplasm, as mentioned in item #3 above, is of particular importance, as compositions and methods of the instant invention are advantageously applied to selective modulation of one population or isoform of a target (e.g., the cytoplasmic isoform) preferentially over another (e.g., the nuclear isoform), providing therapeutic opportunities that would otherwise be implausible due to toxicity, compatibility and/or the essentiality of certain targets, such as nuclear G4R1, for which knockout is embryonically lethal.

Helicases are enzymes that unwind nucleic acids (DNA and/or RNA), which can fold into non-canonical as well as canonical helical structures. Helicases such as G4R1 unwind both DNA and RNA substrates and are therefore recognized as RBPs. In the case of G4R1, unwinding of G4 repeats in target mRNA molecules form G-rich repeat expansions, which results in aberrant translation that yields toxic peptide products implicated in a number of disease processes, including ALS, FTD and Huntington's disease. Other RBPs such as TDP-43 are also implicated in the etiology of neurodegenerative diseases, although the mechanism appears to involve the formation of protein aggregates rather than toxic peptides.

Drug discovery strategies targeting G4R1, TDP-43, and other RBPs include, but are not limited to, small molecule inhibitors, RNAi, ASOs, gene editing, protein-based therapeutics (including antibodies), antibody mimics, such as nucleic acid aptamers, peptides and natural products, such as isolates or purified constituents of plants, fungi or other biological sources.

### Small Molecule Inhibitors

Small molecule inhibitors are compounds of low molecular weight, typically less than 1000 Daltons, which may be isolated from natural sources, selected by combinatorial chemistry or identified by structure-based design, often in combination with molecular modeling and computational chemistry. Structure-based design relies on determining the 3D structure of the helicase enzyme and designing small molecules that can bind to its active site or other critical regions, inhibiting its function. HTS can be used to interrogate large, highly diverse libraries of chemical compounds to identify those that bind to and inhibit the helicase enzyme or influence other RBP targets.

### RNA Interference

RNAi approaches to helicase/RBP targeting include the use of small interfering RNA (siRNA) molecules designed to target the helicase/RBP mRNA or short hairpin RNA (shRNA) constructs that can be delivered to cells to express siRNA molecules continuously.

Alternatively, siRNA molecules can be delivered systemically, advantageously with modifications to enhance uptake and intracellular delivery.

### Antisense Oligonucleotides

ASOs are designed to bind to the helicase-encoding mRNA, preventing its translation into protein. Effective ASO therapy requires efficient delivery vehicles or methods to ensure that ASOs reach and penetrate target cells, including crossing the blood-brain barrier in the case of neurological diseases.

### Gene Editing

Gene editing techniques, such as CRISPR-Cas9 and the litany of recent variants thereof, can be used to precisely edit the gene encoding the helicase enzyme, introducing mutations that disrupt its function.

### Protein-Based Therapeutics

Protein- and antibody-based inhibitors can bind to and inhibit the helicase enzyme, blocking its active site or other critical regions. These inhibitors can be selected by hybridoma technology, in vitro evolution or screening of large collections of natural products or diverse libraries of synthetic compounds.

### Antibody Mimics

Nucleic acid aptamers and synthetic peptides can be selected by in vitro evolution strategies to bind to the target enzyme with high affinity and specificity. These molecules can be further optimized through iterative rounds of selection and amplification.

### Considerations for Therapeutic Development

Key considerations for each of the above-mentioned approaches include:
Specificity: Ensuring that the therapeutic approach targets the specific helicase enzyme involved in unwinding G4 regions of mRNA while minimizing off-target effects.
Toxicity: Minimizing potential side effects, adverse reactions, hypersensitivities and other untoward consequences.
Delivery: Effective administration and targeting methods to ensure that the therapeutic agent reaches the target cells.
Pharmacokinetics: Optimal absorption, distribution, metabolism and excretion properties. Orally available drug discovery is advantageously informed by attention to the Lipinski's rule of five, which is a set of guidelines used in drug discovery to predict whether a chemical compound can be taken orally. Guidelines include (i) molecular weight of less than 500 Daltons; (ii) no more than 5 hydrogen bond donors; (iii) no more than 10 hydrogen bond acceptors; (iv) calculated octanol-water partition coefficient (Clog P) less than or equal to five and (v) a polar surface area less than 140 Å². The rule states that an orally active drug can have no more than one violation of these conditions. The name comes from the fact that all the conditions have multiples of five as the determinant criteria.
Long-term efficacy: Ensuring a long-lasting effect and the prevention of disease progression.
Combination therapies and precision medicine: Combining multiple therapeutic approaches may enhance efficacy and reduce the risk of toxicity or resistance. Precision medicine strategies can tailor the therapeutic approach to the specific genetic and molecular characteristics of each patient or patient cohort.
Monitoring and evaluation: Biomarkers can be used to monitor the effectiveness of the therapeutic approach and assess disease progression.
Clinical development: Developing a potential therapeutic approach into a clinically validated product is a time-, resource-, and capital-intensive process that requires attention to animal models, preclinical study design and well-controlled clinical trials.
General applicability to RBP-directed drug discovery: The general principles described herein for targeting helicases are applicable to a broader range of RBPs implicated in various diseases, although different RBPs operate through distinct molecular and cellular mechanisms. For example, TDP-43, an RBP involved in multiple steps of protein production, is implicated in TDP-43 proteinopathy through a mechanism involving the aggregation of the protein in neurons. TDP-43 proteinopathy is linked to several neurodegenerative disorders, including ALS, FTD and Alzheimer's disease.

### Example 2. NLS-Independent Complementary Nucleotide Therapeutic

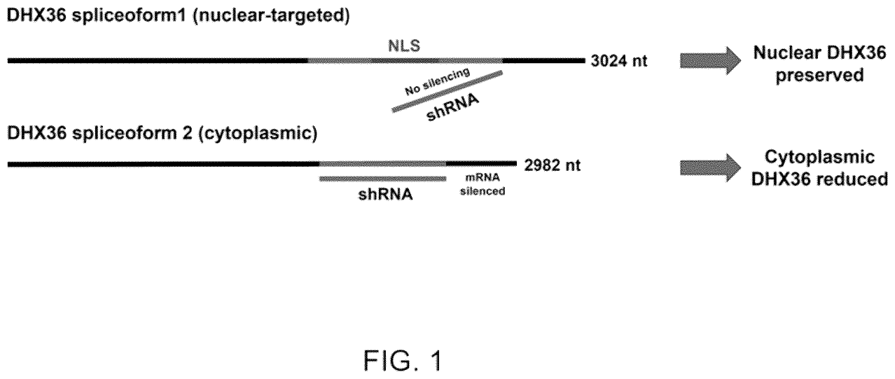

In one embodiment, the present invention includes a composition including a nucleotide therapeutic agent that is complementary to a nucleotide sequence that traverses the splice junction of Spliceoform 2 but does not include a complement to the nuclear localization signal sequence. FIG. 1 illustrates a method and composition for selectively downregulating the cytoplasmic G4 helicase activity of G4R1. shRNA is an example of an embodiment of a therapeutic agent for downregulating cytoplasmic G4 helicase activity of G4R1 by binding to mRNA coding for G4R1. The shRNA is configured to target mRNA sequences adjacent to the nuclear localization signal, such that the shRNA binds to Isoform 2 RNA, resulting in reduced translation of mRNA coding for cytoplasmic G4R1. The shRNA does not bind substantively to Isoform 1 RNA and therefore does not reduce translation of mRNA coding for nuclear G4R1. The cytoplasmic population of G4R1 is thus reduced while the nuclear population of G4R1 is preserved.

Presented below is Table 1, which summarizes general experimental parameters for antisense oligomer targeting of DHX36 Isoform transcripts of multiple species as a demonstration of strategically altering the spatiochemical control network of enzymatic metabolism to alter disease fate or modify cellular physiology.

TABLE 1
Antisense oligomer targeting of human DHX36 Isoform transcripts

| | | | Expected | |
|---|---|---|---|---|
| | | ASO Target | ASO | |
| Gene | Target Isoform Type | Transcipt Region | Mechanism | |
| Target | (Example) | (Conceptual) | (Common) | Notes for ASO Design (General) |
| DHX36 | Isoform 1 (e.g., | Sequence spanning | RNase H- | Target sequence could consist of |
| | includes NLS Exon) | the unique splice | dependent | 15-25 nucleotides, chemically |
| | | junctions including | degradation | modified (e.g., “gapmer” design for |
| | | NLS and 3′ or 5′ | | end stability and RNase H |
| | | exon. | | activation with uniqueness |
| | | | | optimized). |
| DHX36 | Isoform 2 (e.g., | Sequence spanning | RNase H- | Target sequence could consist of |
| | excludes NLS Exon ) | the alternative | dependent | 15-25 nucleotides, chemically |
| | | splice junction | degradation | modified (e.g., “gapmer” design for |
| | | unique to Isoform 2 | | end stability and RNase H |
| | | (where NLS Exon is | | activation). |
| | | skipped) | | |
| DHX36 | Isoform transcript in | Sequence specific | Steric | May use 2′-O-methyl, PNA or LNA |
| | process of splicing | Isoform 2 and the | hindrance | modifications to diminsih splcing |
| | NLS exon | splice donor and | (blocking | out of NLS Exon. |
| | | splce acceptor | translation | |
| | | regions controling | initiation) | |
| | | NLS Exon | | |

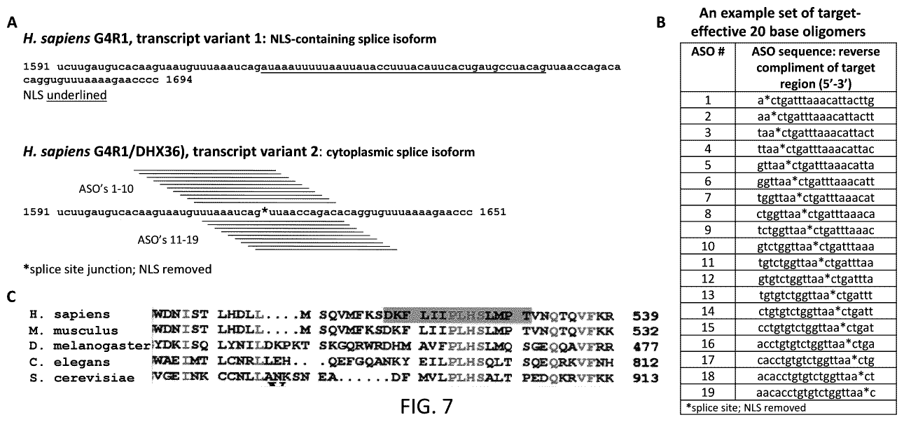

As is apparent from Table 1 and from FIG. 7, the inventors have developed ASO-based approaches to target either (nuclear) Isoform 1 or (cytoplasmic) Isoform 2 of the human G4-helicase DHX36 (aka G4R1). Cytoactive agents of the instant invention are designed and selected to specifically inhibit the cytoplasmic isoform (2) with minimal effect on the nuclear isoform (1) as a therapeutic strategy for treatment of neurodegenerative diseases (e.g., ALS) through a mechanism involving suppression of RAN translation.

Although not set forth in Table 1, similar approaches can be devised for other species based upon appropriately conceived ASO targeting strategies for suitable transcript regions.

FIG. 7. illustrates an example of an isoform-specific experimental strategy to modulate G4R1 (aka DHX36), showing (panel A) human mRNA sequences of G4R1 variant 1 containing an NLS sequence (top) and variant 2 with NLS removed (bottom). ASO's are shown staggered across the NLS splice junction site unique to variant 2. Panel B shows ASO sequences targeted to G4R1 variant 2. Panel C shows that the G4R1 NLS sequence (shaded) is well conserved across humans, mice, flies, worms and yeast (Tran et al.) rendering the above strategy in humans applicable to other species. Cytoactive agents disclosed herein selectively target one or another isoform, cellular compartment or spatiochemical subpopulation of a disease-associated regulatory protein as a means of increasing target discrimination to achieve levels of therapeutic specificity that reduce off-target effects and associated untoward consequences.

The oligonucleotide sequences displayed in FIG. 7 are recited below:

(SEQ ID NO: 1)
actgatttaaacattacttg
(SEQ ID NO: 2)
aactgatttaaacattactt
(SEQ ID NO: 3)
taactgatttaaacattact
(SEQ ID NO: 4)
ttaactgatttaaacattac
(SEQ ID NO: 5)
gttaactgatttaaacatta
(SEQ ID NO: 6)
ggttaactgatttaaacatt
(SEQ ID NO: 7)
tggttaactgatttaaacat
(SEQ ID NO: 8)
ctggttaactgatttaaaca
(SEQ ID NO: 9)
tctggttaactgatttaaac
(SEQ ID NO: 10)
gtctggttaactgatttaaa
(SEQ ID NO: 11)
tgtctggttaactgatttaa
(SEQ ID NO: 12)
gtgtctggttaactgattta
(SEQ ID NO: 13)
tgtgtctggttaactgattt
(SEQ ID NO: 14)
ctgtgtctggttaactgatt
(SEQ ID NO: 15)
cctgtgtctggttaactgat
(SEQ ID NO: 16)
acctgtgtctggttaactga
(SEQ ID NO: 17)
cacctgtgtctggttaactg
(SEQ ID NO: 18)
acacctgtgtctggttaact
(SEQ ID NO: 19)
aacacctgtgtctggttaac

Therapeutic compositions are created by transforming long repeat expansion neuronal progenitor cell lines (e.g., 800 repeats) and short repeat neuronal progenitor cell lines (e.g., 70 repeats) with plasmids encoding shRNA sequences that traverse (but do not include) the nuclear localization signal mRNA flanking sequences (but do not include the NLS sequence). For instance, nucleic acid molecules 10-30 nucleotides in length may be designed to have symmetrical or asymmetrical sequence complementarity to the NLS flanking sequences. A symmetrical 20-nucleotide embodiment of this approach is represented, e.g., by the sequence, GUUUAAAUCAGUUAACCAGA, in which 50% of the nucleotides are complementary to the flanking sequence 3′ to the NLS, while the other 50% of the nucleotides are complementary to flanking sequences 5′ to the NLS. Other embodiments include asymmetrical distribution of complementary sequences across the junction and various approaches to scanning overlapping sequences of fixed length across the junction region.

### Example 3: Inhibitor of the Cytoplasmic Subpopulation of G4R1

In one embodiment, a therapeutic composition for the treatment of a neurodegenerative disease includes an inhibitor of cytoplasmic G4R1 helicase activity and a pharmaceutically acceptable carrier, where the inhibitor is specific for the cytoplasmic G4R1 helicase and does not substantially inhibit the activity of other G-quadruplex helicases or of the nuclear G4R1 Resolvase activity. In some embodiments, the neurodegenerative disease is ALS, FTD, fragile X, fragile X tremor ataxia syndrome (including other guanine-rich fragile sites), myotonic dystrophy type 2, spinocerebellar ataxia, cerebellar ataxia neuropathy, vestibular areflexia syndrome, myoclonic epilepsy, oculopharyngeal muscular dystrophy and myotonic dystrophy type 1.

In one embodiment, the therapeutic composition includes a neuroprotective agent, e.g., a free radical scavenger, antioxidant or anti-inflammatory agent to help reduce inflammation in the nervous system.

In another embodiment, the therapeutic composition is formulated in a manner that facilitates transport across the blood-brain barrier, e.g., through the inclusion of lipophilic appendages or permeabilizing agents.

In one embodiment, the therapeutic composition includes at least one of a pharmaceutical carrier, a membrane delivery system or an agent that alters the cellular distribution of the cytoplasmic isoform of G4R1. G4R1 reduction has been shown to reduce cell cycle progression and promote oncogenesis, and, as such, another embodiment of this invention is a therapeutic composition and corresponding methods for the treatment of oncogenic diseases.

### Example 4: Cytoplasmic Helicase Inhibitor Selection

In one embodiment, molecules that specifically bind to the cytoplasmic isoform of G4R1 but do not bind to the nuclear isoform of this enzyme are selected in the following way: two populations are created, each pure for a single isoform, e.g., without limitation, by the recombinant expression of their DNA sequences. These two populations are used as binding targets for combinatorial libraries of different classes of molecules, including but not limited to, oligonucleotides, modified oligonucleotides, peptides and small molecules. Specific methodological examples of such techniques include SELEX, which can be used for selecting oligonucleotide aptamers, and phage display, which can be used as a means to select binding peptides. Various binding assays advantageously including homogeneous assays leveraging techniques like FRET signaling are used to identify small molecule binding from diverse libraries of low molecular weight compounds, preferably libraries designed with favorable absorption, distribution, metabolism and excretion (ADME) properties. Molecules that specifically bind the cytoplasmic isoform of G4R1 and that do not bind the nuclear isoform of G4R1 can be thus selected. Such molecules from these libraries that are specific for the cytoplasmic protein can then be screened or modified to induce inhibition of cytoplasmic G4R1 helicase activity. Some examples of means for inhibiting the cytoplasmic enzyme activity are molecular binding leading to direct inhibition at the catalytic site of the enzyme, binding that changes the normal localization of the enzyme or binding that increases protein turnover, among other means well known in the art.

In one embodiment, the present invention includes selection methods for compounds that inhibit helicases in specific, user-defined cellular compartments or locations, while sparing members of the same class of helicases in other cellular compartments or locations. These compounds are selected from biased (aka targeted) screening libraries specifically tailored to identify helicase-binding ligands. An exemplary helicase screening library is available from Life Chemicals, containing more than 7,000 drug-like, helicase inhibitor candidates. As described elsewhere, and using cytoplasmic vs. nuclear discrimination as an example, inhibitors that act only on cytoplasmic helicase populations and not nuclear helicase populations are identified. To assess the effectiveness of each compound in the library, cells are grown in a high-throughput tissue culture format (e.g., 96-well plates) incorporating ligand-dependent, conformationally switchable FRET-labeled G-quadruplex DNA or RNA and a compound from the screening library. A set of control samples is treated with G-quadruplex DNA or RNA (or other helicase substrates of interest) and control vehicle (no drug). The FRET-labeled DNA or RNA comprises donor (e.g., FITC [fluorescein isothiocyanate] with 498/517 nm absorption/emission peaks) and acceptor (e.g., TAMRA [carboxytetramethylrhodamine] with 552/578 nm absorption/emission peaks) designed to emit a fluorescent signal from the terminal acceptor fluorophore [TAMRA] following helicase-dependent unwinding of the FRET pair-labeled quadruplex in a manner that disrupts energy transfer from donor to acceptor as a result of increased distance between members of the FRET pair due to unwinding of the quadruplex. The FRET signal is measured using an Agilent BioTek Synergy H1 Hybrid Multi-Mode Reader with excitation at around 490 nm and emission at around 550 nm in experimental and control wells, and changes in relative FRET are compared. Candidate compounds that result in altered relative FRET signaling between experimental and control wells for each compound are further assessed with fluorescent microscopy analyses to assess which compounds result in FRET changes primarily in one cell compartment and not another (e.g., in the cytoplasm and not in the nucleus). Additionally, compounds from the library that show helicase inhibition may be further modified to restrict their localization to specific cell compartments or locations through multifactorial optimization of candidate signaling and cellular distribution properties. Other embodiments of the invention are explicitly directed to the compounds themselves, including selected compositions of matter, applications and methods of making and using selected compositions for therapeutic applications disclosed throughout the specification.

### Example 5: Treatment Method Targeting G4R1 mRNA

In another embodiment, the present invention includes a method for treating a neurodegenerative or oncogenic disease, including administering to a subject an agent configured to bind mRNA coding for the protein G4R1 and lacking a nuclear localization signal. In some embodiments, the agent is configured to be capable of binding mRNA coding for protein G4R1 and avoiding a nuclear localization signal. In some embodiments, the neurological disorder is amyotrophic lateral sclerosis, frontotemporal dementia, Huntington's disease, Fragile X syndrome, ataxia, a polyglutamic disorder or a repeat expansion disorder. In another embodiment, a therapeutic composition is used for the treatment of cancers. In some embodiments, the agent is an oligonucleotide agent. In some embodiments, the oligonucleotide agent is shRNA. In some embodiments, the subject is a human subject.

### Example 6: Customizable Research Kits for Compartment-Selective Modulation of Cellular Enzyme Activity

Kits are provided herein for the selective inhibition or activation of one targeted enzyme subpopulation or another without substantive alteration of other isoforms or subpopulations of the enzyme. Kits include a pool of customizable RNAi and/or antisense oligonucleotides (ASO) that targets a single cohort, e.g., the cytoplasmic enzyme population, while sparing the nuclear population of the target enzyme or, conversely, targets nuclear enzyme and spares the cytoplasmic subpopulation or customizable oligos that specifically block the out-splicing of the nuclear localization site or enhance the ability to slice out the nuclear localization site. The kit includes transfection reagents to deliver the RNAi and/or ASOs, as well as nuclear/cytoplasmic fractionating reagents and, in one mode of operation, marker antibodies to distinguish nuclear and cytoplasmic enzyme populations or isoforms. These antibodies are used to confirm successful fractionation of the nuclear and cytoplasmic compartment targets.

Research kits are also contemplated for the purification of subcellular enzyme fractions, e.g., for purifying nuclear and cytoplasmic protein fractions in a manner that preserves native protein structure and function in resulting isolates.

### Example 7: Library Screening Approaches to Identify RNAi Sequences for Subpopulation-Specific Enzyme Inhibition

With the purpose of screening and selecting of RNAi sequences that reduce either cytoplasmic or nuclear subpopulations of a target enzyme but not both subpopulations, methods are presented here for identifying suitable RNAi candidates. An exemplary approach is to design RNAi molecules to target sequences that flank the nuclear localization but do not include the NLS sequences or target—only the NLS sequence. A pool of candidate sequences complementary to approximately 18-30 nt targeting this region are prepared via commercial oligonucleotide synthesis designed to either symmetrically or asymmetrically span the junction. Following RNAi treatment, cells are harvested, and nuclear and cytoplasmic fractions isolated. Western blot analysis is performed on each fraction and compared to control (mock-treated) cells. Alternatively, a cell line in which the target enzyme is genomically tagged with green fluorescent protein (GFP) is generated and treated with RNAi as above. Changes in nuclear and cytoplasmic abundance and localization are determined using fluorescence microscopy.

### Natural Products

Natural product libraries are screened and selected using a 96-well plate format (Screen 1). Natural product libraries are first screened for ability to inhibit helicase activity. Activity is monitored with a DNA or RNA substrate. The substrate is labeled in a way that allows for a detectable change following catalytic action upon substrate. For example, in one embodiment a tetramolecular G-quadruplex substrate is converted to monomers following a reaction designed to enhance fluorescence signaling through FRET. The reaction includes the labeled substrate tube, recombinant helicase and a natural product. The enzyme activity is measured via changes in substrate signal compared to control samples. These steps are repeated for each compound in the chosen molecular library which may include natural product libraries, natural product-like libraries, bioactive libraries, drug-like compound libraries, fragment libraries or similar libraries.
Screen 2A. Once candidates are identified that inhibit target enzyme activity (Screen 1), then these candidates are screened for ability to inhibit cytoplasmic enzyme activity while sparing nuclear target enzyme activity and vice versa. To do this, recombinant cytoplasmic and nuclear G4R1 are prepared and screened for preferential inhibition.
Screen 2B. Alternatively, the candidate compounds shown to inhibit enzyme activity in Screen 1 are modified in a way that renders them permeable to the plasma membrane but excluded from the nucleus.
Screen 3. Once candidates have passed Screens 1 and 2, candidate molecules are tested to confirm reduced RAN translation of toxic dipeptide repeats using western blots and luciferase activity assays for RAN translation protein products.

Methods of preparing reagents are summarized below.

### Cell Culture

For cell culture, cell lines are cultured and passaged at 37° C., 5% CO₂. Cells are maintained in Dulbecco's modified Eagle's medium supplemented with 10% fetal bovine serum, 1% nonessential amino acids and/or other cell line appropriate reagents.

### Analytical Methods

Demonstration of effective reduction of cytoplasmic G4R1 is achieved as follows:

### G4 Activity Assays

G-quadruplex helicase activity is determined as previously described (Vaughn et al., JBC) but in a modified RES buffer (K-Res, 100 mm KCl, 10 mm NaCl, 3 mm MgCl₂, 50 mm Tris acetate, pH 7.8, 70 mm glycine, 0.012% bovine α-lactalbumin, 10% glycerol). 0.2 pmol of 5′-end-labeled G-quadruplex (³²P or TAMRA) are included per 50-μl reaction. Reactions are allowed to proceed at 37° C. for 30 min, stopped by addition of 5 μl of 200 mm EDTA, and analyzed by electrophoresis through a 10% non-denaturing polyacrylamide TBE gel with 10% glycerol. Next, 25 μl of reaction mixture containing 1 unit of recombinant enzyme is added (50 μl total reaction volume) at 4° C., then reaction mixtures are incubated at 37° C. for 30 min, dropped to 4° C., and stopped with 5 μl of 200 mm EDTA. Reactants are separated by TBE 2% agarose gel electrophoresis. Gels are scanned on a Typhoon 9210 Imager (GE-Healthcare, Chicago, IL) and images analyzed.

### Western Blots

In a 12-well plate, cell lines are rinsed with 500 μl cold 1×PBS twice and then lysed in 300 μl radioimmunoprecipitation buffer (RIPA) with protease inhibitor (120 μl for 24-well plates) for 30 min in a 4° C. shaker. Lysates are homogenized by passing through a 28-gauge syringe eight times, mixed with 6× sample buffer with a final concentration of 2% beta-mercaptoethanol (β-ME), denatured at 95° C. for 10 min, and stored at −20° C. Protein samples are standardized by bicinchoninic acid assay for equal total protein loading. About 20 μl of each protein sample is loaded in each well of a 10% SDS-PAGE, subjected to electrophoresis and then transferred to a nitrocellulose or equivalent substrate. All primary antibodies applied for Western blot are used at 1:1000 dilution or manufacturer's recommendations in 5% nonfat dairy milk (w/v) and 0.1% Tween-20 (v/v) in Tris-buffered saline except antipuromycin at 1:5000 dilution. The following antibodies are used, anti-DHX36 (aka anti-G4R1) from Proteintech (13159-1-AP), anti-GAPDH from Santa Cruz Biotechnology (catalog no. sc-32233), which is used as a marker of cytoplasmic fraction, and anti-Lamin B1, which is used as a nuclear fraction marker.

### Luciferase Activity Assays for Assessing RAN Translation Products

For transfection and luciferase assay cells are plated in 96-well plates at 2.5×10⁵ cells/well in 100 μl media. About 24 h after plating, cells are cotransfected with 50 ng/well of pcDNA (+)-NLuc-3×FLAG plasmids and 50 ng/well of pGL4.13 FFLuc plasmid as transfection control. Transfection is performed using Viafect transfection reagent (Promega; catalog no. E4981) with mixed plasmids dropwise in cultured cells after 10 min of incubation at room temperature and then gently shaking the plate for 1 min. Plasmid DNA and C9-repeat RNA cotransfection are performed by forward transfection of published DNA plasmid expressing empty vector, WT, or E335A DHX36 (41) in HeLa cells seeded at 2.5×10⁵ cells/well in 100 μl media. After 24 h, in vitro-synthesized C9-RNA and pcDNA-FF RNA are cotransfected at 50 ng/well each into the well by Viafect transfection reagent (Promega; catalog no. E4981) as described previously. Luciferase assays are performed 48 h after plasmid transfection. Cells from each well are collected in microcentrifuge tubes, and media removed after 400 rpm centrifugation for 5 min. Then cells from each tube are lysed with 60 μl of Glo Lysis Buffer 1× (Promega; catalog no. E2661) and vortexed for 5 s. In opaque white 96-well plates, 25 μl from the 60 μl of cell lysate are distributed to mix with 25 μl of Nano-Glo Luciferase Assay System (Promega; catalog no. N1120), and another 25 μl of cell lysate is mixed with 25 μl of ONE-Gbo Luciferase Assay System (Promega; catalog no. E6130). The plate is placed on a shaker for 5 min in the dark. Luciferase activity in each well is determined by luminescence measurements. All reagents, experiments and resulting data are presented at room temperature.

### Example 8: Antisense Oligonucleotides for the Treatment of Neurodegenerative Diseases

In one embodiment a drug for the treatment of ALS or FTD, cancers or related diseases is designed with a sequence specifically targeting the cytoplasmic isoforms of enzymes that also have nuclear isoforms that harbor nuclear localization signals, such as G4R1 (aliases DHX36 and RHAU). Spliceoform 2 mRNA is targeted by hybridization overlapping the unique junction sequence formed in Spliceoform 2 RNA from the removal of the NLS. The molecule is designed to stably bind Spliceoform 2 mRNA while not binding appreciably to Spliceoform 1 mRNA. Hybridizing targeting oligomers from 12-40 bases are made of DNA or of synthetic, nonbiologically derived binding mimetics of DNA or RNA such as constructs including but not limited to the following: 2′-O-methyl oligonucleotides, phosporothioate oligonucleotides, 2′-O-methoxyenthyl oligonucleotides, locked nucleotides, peptide nucleic acids and other nucleotide analog-based oligonucleotides known in the art. A symmetrical 20-nucleotide embodiment of an antisense approach is represented by the base sequence, GUUUAAAUCAGUUAACCAGA, in which 50% of the nucleotides are complementary to the flanking sequence 3′ to the NLS, while the other 50% of the nucleotides are complementary to flanking sequences 5′ to the NLS. Oligonucleotides may also be created with chimeras of different types of these molecules to maximize stability and in some cases catalytic activity. These compounds may also be capped on the 5′ and 3′ ends with modifications to improve stability as well as to enhance delivery into the cell. The antisense compound can be administered to the patient as a prophylactic to protect against neural damage or in more advanced disease to slow or halt cell degeneration. The compound can be administered at a dosage to create a cytoplasmic concentration one order of magnitude higher than its dissociation constant (Kd) for binding. For example, 3-1000 mg of the compound may be administrated intrathecally or by other means including, but not limited to, intracerebroventricular injection, intranasal delivery, oral delivery or systemic intravenous delivery. In one iteration, three doses are administered every 14 days, followed by a dose 30 days after the third dose, and then one dose every four months. This protocol can potentially be continued for the life of the patient.

### Example 9: Inhibitor of RNA Binding Proteins Mis-Localized in Disease

In one embodiment, a drug is designed to specifically bind a mis-localized population of an RNA binding protein but not to bind the correctly localized population of the RNA binding protein. Illustrative examples include RNA binding proteins TDP-43 and FUS, which are commonly mis-localized from the nucleus to the cytoplasm in neurodegenerative diseases such as ALS and FTD. In these examples, drugs are designed to inhibit, reduce or re-localize cytoplasmic TDP-43 or FUS and not inhibit, reduce or re-localize nuclear TDP-43 or FUS. Another embodiment includes drugs designed to specifically bind to subpopulations of RBPs that are destined for or already mis-localized within subcellular organelles or compartments such as stress granules, nucleoli, Cajal bodies or P-bodies. Mis-localized populations are specifically targeted using RNAi, antisense oligonucleotides, small molecules, peptides, natural products, phage display molecules, antibodies or other similar approaches. An exemplary means of targeting mis-localized TDP-43, FUS or other RNA binding proteins involves targeting Spliceoforms/isoforms that contain a nuclear export sequence (NES) or NLS to reduce or otherwise inhibit cytoplasmic protein. Some examples of means for targeting the mis-localized proteins are molecular binding leading to direct inhibition of protein-protein interactions, binding that changes the localization of the enzyme, binding that reduces protein synthesis or binding that increases protein turnover, among other means well known in the art.

In one embodiment, molecules that specifically bind to the cytoplasmic isoform of the enzyme G4R1 but do not bind to the nuclear isoform of the enzyme G4R1 are selected in the following way: two populations are created, each pure for a single isoform, e.g., without limitation, by the recombinant expression of their DNA sequences. These two populations are used as binding targets for combinatorial libraries of different classes of molecules including, but not limited to, oligonucleotides, modified oligonucleotides, peptides and small molecules. Specific methodological examples of such techniques include SELEX, which can be used for selecting oligonucleotide aptamers, and phage display, which can be used as a means to select binding peptides. Various binding assays, including homogeneous assays assisted by FRET signaling are used to identify small molecule binding from diverse libraries of low molecular weight compounds, preferably libraries designed with favorable ADME properties. Molecules that specifically bind the cytoplasmic isoform of G4R1 and that do not bind the nuclear isoform of G4R1 can be thus selected. Such molecules from these libraries that are specific for the cytoplasmic protein can then be screened or modified to induce inhibition of cytoplasmic G4R1 helicase activity. Some examples of means for inhibiting the cytoplasmic enzyme activity are molecular binding leading to direct inhibition at the catalytic site of the enzyme, binding that changes the normal localization of the enzyme or binding that increases protein turnover, among other means well known in the art.

### Example 10: Isoform-Selective Modulation of Proteins for Studying or Treating Cellular Disorders

Compositions of the present invention are designed to modulate cellular functions through isoform-specific interactions with applications in cell culture, tissue and organ studies, and in vivo therapeutics. These compositions are particularly useful for drug discovery, disease mechanism studies, regenerative medicine and human or veterinary therapeutics that rely on isoform-selective targeting. Isoform-specific modulation of NABPs, which include RBPs and DBPs, is a promising approach for treating cellular disorders. NABPs play critical roles in various cellular processes such as transcription, translation, intracellular transport and RNA metabolism. Dysregulation of these proteins is implicated in a wide range of diseases, including cancer, neurodegenerative disorders and genetic diseases.

RBPs such as TDP-43, FUS, TIA-1, hnRNP A1 and G4R1 are involved in essential processes like RNA splicing, transport and stress granule formation. Mutations and/or dysregulations of these proteins lead to the formation of toxic protein aggregates, which contribute to diseases like ALS, FTD and various cancers, including lung and breast cancers. TDP-43 and FUS are linked to neurodegenerative diseases, with mutations causing toxic aggregate formation in neurons. hnRNP A1 dysregulation is associated with cancer and promotes tumorigenesis by disrupting gene expression. TIA-1 is implicated in stress response dysfunction and is linked to neurodegenerative diseases and viral infections.

As mentioned elsewhere, DBPs such as p53, BRCA1, BRCA2, CTCF and CNBP are central to gene regulation and chromatin organization. Mutations in these proteins are frequently associated with cancers and genetic disorders. p53, a tumor suppressor, is mutated in many cancers. BRCA1 and BRCA2 mutations are linked to hereditary breast and ovarian cancers. CTCF regulates chromatin structure, and its dysfunction is associated with various cancers. CNBP, implicated in transcription and translation regulation, is associated with myotonic dystrophy type 2. The present invention is not directed to the targeting of mutant proteins, per se, but rather to protein variants referred to herein as isoforms, subpopulations, populations or Spliceoforms.

The dysregulation of NABPs is a contributing factor in many diseases, underscoring the importance of these regulatory proteins as therapeutic targets. However, a challenge in targeting these proteins is that many isoforms are essential for cellular viability, as shown by knockout studies. Thus, isoform-selective therapies as disclosed herein offer a strategy to selectively modulate these proteins without disrupting their essential functions. The present invention focuses on isoform-specific targeting of NABPs to avoid the toxicity and cell death associated with global knockout or inhibition. This approach can be applied to diseases such as neurodegenerative disorders like ALS and FTD, cancers and genetic diseases.

### Example 11: Application of Isoform-Specific Compositions and Methods to Cellular Proteins with a Focus on DNA-Binding Proteins

DBPs play critical roles in regulating the genome, maintaining cellular integrity and controlling cellular responses to environmental stressors such as DNA damage. Mutations or dysregulation of these proteins can lead to a variety of diseases, including cancer and genetic disorders. The present invention applies isoform-specific targeting to DBPs, providing a method for selectively modulating the function of these proteins in highly specific manner to treat or prevent diseases associated with protein dysregulation and/or dysfunction.

p53 is a key tumor suppressor protein often referred to as the guardian of the genome due to its central role in regulating the cell cycle and initiating DNA damage response pathways. Mutations in p53 are found in over 50% of human cancers, resulting in loss of its tumor-suppressive activity and allowing uncontrolled cell growth. Not only is p53 subject to mutation, this protein also exists in different isoforms that result from alternative splicing, promoter usage and translation initiation of the p53 gene. Isoform-selective modulation of p53 offers a targeted approach to restore its function in cancer cells, thereby preventing tumor formation and progression without disrupting the normal function of other isoforms required for healthy cellular processes. Through isoform-specific interventions, the present invention can selectively activate p53's tumor-suppressive functions in cancerous tissues, while avoiding the deleterious effects of broad p53 activation, which can cause toxicity in non-cancerous cells.

Unlike efforts to target disease-associated cell surface antigens and biomarkers comprising mutant proteins such as BRAF mutations (found in melanomas and colorectal cancers), BRCA mutations (associated with breast, ovarian and prostate cancers), EGFR mutations (e.g., in non-small cell lung cancer), HER2 mutations (found in various cancers, including breast and lung) and P53 mutations (common in breast cancer and associated with more aggressive tumors), the instant invention is directed toward isoform-specific protein targeting as distinct from historic approaches to address pathologies associated with mutant proteins. Subpopulations of expressed gene products of the instant invention are not mutations, but spatiochemical and spatiotemporal variants that enable precision targeting through novel compositions and methods disclosed herein.

CTCF is a DNA-binding protein involved in chromatin organization, gene regulation, and the insulation of genomic domains. Disruptions in CTCF function have been linked to a variety of cancers and developmental disorders. Aberrant CTCF binding or dysfunction can lead to altered gene expression patterns, contributing to disease development. The isoform-specific modulation of CTCF presents a novel approach for addressing these disruptions by selectively restoring or modulating CTCF activity at specific loci without affecting its broader chromatin-related functions. This targeted intervention can help prevent or reverse gene misregulation in diseases driven by CTCF dysfunction.

In addition to the well-known DBPs like p53, BRCA1, BRCA2 and CTCF, other NABPs such as CNBP and PABP are also implicated in human diseases and can be targeted through isoform-specific strategies. CNBP is involved in various cellular processes, including transcription and translation, and mutations in CNBP are associated with myotonic dystrophy type 2 (DM2), a neuromuscular disorder characterized by muscle weakness and myotonia. Isoform-specific targeting of CNBP can help correct transcriptional and translational dysfunction in affected tissues, providing a potential therapeutic approach for DM2. PABP, which plays a key role in mRNA stability and translation, has been linked to various diseases, including cancer and neurodegenerative disorders. Targeting specific isoforms of PABP can restore normal mRNA metabolism and translation, improving cellular function and alleviating disease symptoms.

This example illustrates areas of application of the invention as a highly specific and effective method for modulating DBPs and other NABPs by targeting individual isoforms in cell-based compositions as a window to understanding of cellular mechanisms for use in drug discovery and development of isoform-specific therapeutics. By focusing on the isoforms most relevant to disease processes, these methods and corresponding compositions can provide therapeutic benefits while minimizing potential side effects associated with global protein modulation. This approach can be applied not only to studying and reversing cellular disorders in vitro, but also to design, selection and development of therapeutic compositions for a wide variety of diseases, including cancers, immune deficiencies, genetic and cellular disorders and degenerative diseases with applications across both human and nonhuman species.

### Example 12: Isoform-Specific Compositions and Methods for Treating Diseases of Nonhuman Animals

NABPs are critical regulators of cellular processes in both humans and nonhuman animals. These proteins, including RBPs and DBPs, play essential roles in the regulation of gene expression, RNA metabolism, transcription and translation. Dysregulation of NABPs can result in cellular dysfunction and has been implicated in a variety of diseases across many species. The compositions and methods described in the present invention focus on isoform-specific modulation of NABPs as a strategy for treating diseases in nonhuman animals as well as human diseases ranging from neurodegenerative disease to cancers, immune diseases and cellular, genetic and developmental disorders. The instant example is explicitly focused on diseases of nonhuman animals.

RBPs such as TDP-43, FUS, hnRNP A1 and TIA-1 have been implicated in a range of diseases in nonhuman animals. TDP-43 and FUS, for instance, are involved in the accumulation of misfolded protein aggregates in diseases like BSE in cattle and chronic wasting disease (CWD) in cervids such as deer and elk. In both BSE and CWD, the aggregation of TDP-43 and FUS contributes to neurodegeneration and cellular damage. The isoform-selective modulation of these RBPs can offer a therapeutic approach to prevent or reduce the formation of these toxic aggregates. By targeting specific isoforms of TDP-43 and FUS that contribute to disease pathology, the compositions of the present invention can help mitigate neurodegeneration in animals affected by these diseases.

hnRNP A1 has been implicated in various cancers in nonhuman animals. In particular, in cats, hnRNP A1 dysregulation has been associated with feline leukemia virus (FeLV)-induced lymphoma. Isoform-selective targeting of hnRNP A1 offers a potential strategy to restore normal function of this RBP in affected tissues, reducing tumorigenesis and promoting cellular homeostasis in animals with lymphoma or other cancers. By selectively modulating the isoforms of hnRNP A1 that are involved in cancer progression, it is possible to treat or prevent the development of FeLV-induced lymphoma in cats and potentially similar cancers in other species.

TIA-1 is another RBP involved in RNA metabolism and cellular stress responses. Although not extensively studied in animal models, TIA-1's role in stress granule formation and RNA regulation suggests potential links to diseases in animals, particularly those related to neurodegeneration and immune dysfunction. Isoform-specific modulation of TIA-1 offers therapeutic benefits for animals suffering from diseases where stress response pathways are disrupted, offering a new avenue for research and treatment in species affected by stress-related disorders or viral infections.

CTCF, a DNA-binding protein involved in chromatin organization and gene regulation, has been implicated in various cancers and developmental disorders. Disruptions in CTCF function in animals can lead to altered gene expression and contribute to the development of disease. By selectively modulating the isoforms of CTCF that are involved in specific genomic processes, the present invention provides a strategy to correct misregulation and prevent disease in nonhuman animals. This approach can be applied to treat cancers or developmental disorders in animals where CTCF dysfunction is a contributing factor.

In addition to the well-known RBPs and DBPs, other NABPs such as CNBP and PABP may also play a role in diseases of nonhuman animals. CNBP has been associated with transcriptional and translational regulation, and while its role in animal diseases has not been extensively studied, its involvement in cellular processes suggests it may be relevant to a variety of diseases. Isoform-specific modulation of CNBP offers potentially dramatic therapeutic benefits for animals suffering from diseases like myotonic dystrophy type 2 and other disorders involving translational dysfunction.

Similarly, PABP, which regulates mRNA stability and translation, has been linked to various human diseases, and mutations in PABP potentially contribute to corresponding diseases in animals. Targeting specific isoforms of PABP offers the potential to restore normal RNA metabolism and translation, improving cellular function and alleviating symptoms of diseases in animals.

The compositions and methods of the present invention offer a highly specific approach for treating diseases in nonhuman animals through isoform-specific targeting of NABPs. By focusing on the isoforms most relevant to the disease processes in each animal species, these methods can provide therapeutic benefits while minimizing the risk of off-target effects. This isoform-selective strategy can be applied to a wide variety of diseases in nonhuman animals, including cancers, neurodegenerative disorders and genetic diseases, with broad applicability across species including companion animals, livestock and wildlife.

### Example 13: Isoform-Specific CRISPRa-Mediated Upregulation of Nuclear G4R1 Population in Mouse Neurons

CRISPR activation (CRISPRa) is employed to selectively upregulate the nuclear isoform of G4R1 helicase in vitro in mouse neuronal cells. The approach leverages use of a modified CRISPR-Cas system targeting gene-specific enhancers to increase nuclear G4R1 expression while minimizing cytoplasmic isoform upregulation. As noted elsewhere herein, G4R1 is a helicase involved in RNA metabolism having nuclear and cytoplasmic isoforms exhibiting distinct functions. Dysregulation of G4R1 has implications in neurodegenerative diseases. Knockout studies indicate that the nuclear isoform is an essential regulatory protein. The goal of this set of experiments is to increase nuclear G4R1 activity as part of an overall effort to show both up- and downregulation of at least two different isoforms of a particular NABP.

Methodological note: For isoform-specific upregulation in this case, targeting requires a safe-harbor region like ROSA and the use of guide RNA sequence(s) that cleave(s) in that region. cDNA of the gene of interest comprising the NLS is co-transfected with the guide RNA. An alternative option is to knock out splice acceptor and donor regions in the two introns flanking the NLS.

The data indicates that CRISPRa can selectively upregulate the nuclear G4R1 isoform in mouse neuronal cells, demonstrating feasibility for isoform-specific gene regulation. This study provides a foundation for investigating G4R1's role in neurodegenerative diseases and potential therapeutic applications.

### REFERENCES

Konermann et al. (2015). Genome-scale transcriptional activation by an engineered CRISPR-Cas9 complex. Nature, 517(7536), 583-588.
Qi et al. (2013). Repurposing CRISPR as an RNA-guided platform for sequence-specific control of gene expression. Cell, 152(5), 1173-1183.

### Example 14: Experimental Strategies to Modulate TDP-43 Isoforms in Neuronal Cells of Sprague-Dawley Rats

This study explores experimental strategies for specifically modulating nuclear and cytoplasmic TDP-43 isoforms in Sprague-Dawley rat neuronal cells. Transactive response DNA-binding protein 43 (TDP-43) is an RBP essential for RNA metabolism. Dysregulation of TDP-43 has implications in neurodegenerative diseases, such as ALS and FTD.

### Materials and Methods

### Neuronal Cell Culture

1. Isolate peripheral nerve cells from Sprague-Dawley rats.
2. Culture cells in Neurobasal Medium supplemented with 2% B27 and 1% penicillin-streptomycin.
3. Maintain cells at 37° C., 5% CO₂.

### Experimental Strategies

### Upregulation of Cytoplasmic TDP-43 Isoform

Transfect cells with cytoplasmic TDP-43-specific expression vector (pCMV-cTDP-43).
Use lipofectamine 3000 for transfection.
Validate upregulation using RT-qPCR and Western Blot.

### Downregulation of Cytoplasmic TDP-43 Isoform

1. Transfect cells with cytoplasmic TDP-43-specific shRNA vector (pLKO-cTDP-43).
2. Use lipofectamine 3000 for transfection.
3. Validate downregulation using RT-qPCR and Western Blot.

### Upregulation of Nuclear TDP-43 Isoform

1. Transfect cells with nuclear TDP-43-specific expression vector (pCMV-nTDP-43).
2. Use lipofectamine 3000 for transfection.
3. Validate upregulation using RT-qPCR and Western Blot.

### Downregulation of Nuclear TDP-43 Isoform

1. Transfect cells with nuclear TDP-43-specific shRNA vector (pLKO-nTDP-43).
2. Use lipofectamine 3000 for transfection.
3. Validate downregulation using RT-qPCR and Western Blot.

### Validation Methods

1. RT-qPCR: Measure TDP-43 isoform-specific mRNA levels.
2. Western Blot: Evaluate TDP-43 protein levels using isoform-specific antibodies.
3. Immunofluorescence: Visualize subcellular localization of TDP-43 using confocal microscopy.

### Outcomes

1. Successful upregulation/downregulation of cytoplasmic and nuclear TDP-43 isoforms.
2. Validation of isoform-specific modulation using RT-qPCR, Western Blot, and immunofluorescence.

### Discussion

This study provides a comprehensive approach to specifically modulate TDP-43 isoforms in Sprague-Dawley rat neuronal cells. Understanding the regulation of TDP-43 isoforms sheds light on their roles in neurodegenerative diseases and potential therapeutic applications.

### REFERENCES

Wegorzewska et al. (2009). TDP-43 and FUS RBPs in frontotemporal dementia and amyotrophic lateral sclerosis. Biochemistry, 48(19), 4078-4087.
Polymenidou et al. (2011). Long pre-mRNA depletion and RNA missplicing contribute to neuronal vulnerability from loss of TDP-43. Nature Neuroscience, 14(4), 459-468.

### Example 15: Naturally Occurring Isoform-Selective Ligands Against NABP for Use as Reference to Enable Molecular Modeling and Computational Chemistry Approaches to Target-Directed Design of Synthetic Ligands

While a comprehensive list of naturally occurring ligands that specifically target NABP isoforms has not been reported, a few examples of isoform-specific NABPs do exist, including:
1. Hormone-receptor interactions: Steroid hormones (e.g., estrogen, testosterone) bind to nuclear receptors, which are ligand-activated transcription factors. Different receptor isoforms exhibit varying hormone affinities, leading to distinct downstream effects. Thyroid hormones bind to thyroid hormone receptors (TRs), leading to tissue-specific responses due to isoform-specific ligand-binding affinities.
2. RBPs recognize specific RNA sequences or structures. Different RBP isoforms can bind to distinct RNA targets, regulating specific cellular processes.
3. Post-translational modifications: Phosphorylation and acetylation can alter NABP conformation and binding properties, affecting ligand and protein interactions.

While there isn't a specific ligand targeting NLS-containing NABPs exclusively, modulating nuclear import can have therapeutic implications. The NLS is a short amino acid sequence that directs proteins to the nucleus. It interacts with importin proteins, facilitating nuclear transport.

### Strategies to Modulate Nuclear Import: Small Molecule Inhibitors

Low molecular weight ligands (aka small molecules) having a molecular weight less than about 1000 Daltons can be designed to bind to the NLS or importin proteins, as can relatively low molecular weight peptides and oligonucleotides, blocking their interaction and preventing nuclear import. Peptide-based inhibitors include peptides that can be synthesized to mimic the NLS sequence or the importin-binding site, competing with endogenous proteins for binding and inhibiting nuclear import. RNAi can be used to knockdown the expression of genes encoding NLS-containing proteins or importin proteins, thereby reducing their levels and inhibiting nuclear import. Protein engineering can be used to alter the amino acid sequence of the NLS or the importin-binding site, making it possible to create mutant proteins that are no longer recognized by the transport machinery, preventing their nuclear localization. While there isn't a specific, naturally occurring ligand for NLS-lacking NABPs, several strategies can be applied to the selection of candidates with desired specificity, including, e.g., combinatorial chemistry, computational chemistry and rational drug design. By leveraging advanced computational tools and structural biology techniques, it is possible to identify unique structural features of isoforms lacking an NLS. This information can be used to design ligands that specifically interact with these isoforms, minimizing off-target effects. HTS enables large-scale screening of chemical libraries comprising low molecular weight candidates that can be used to identify potential ligands that bind selectively to isoforms without an NLS. This approach can be accelerated by using advanced screening technologies, such as fragment-based drug discovery and virtual screening. Peptide-based therapeutics and peptide mimetics leverage the well-traveled methodologies that have been used to design molecular structures that mimic the binding sites of natural ligands or to interfere with protein-protein interactions, affecting the function of specific isoforms. Peptide-based therapeutics offer several advantages, including high specificity, low toxicity, and ease of synthesis. RNAi can be used to selectively target the mRNA of specific isoforms, reducing their expression levels. This approach can be particularly useful for targeting isoforms that are involved in disease processes. Protein engineering principles can be used to modify the amino acid sequence of a protein, making it possible to create new binding sites for specific ligands or to disrupt existing binding sites. This approach can be used to generate protein variants with altered ligand-binding properties. By combining rational drug design, high-throughput screening and other innovative approaches, it is now possible to design and select targeted therapeutic compositions with the ability to effectively modulate the activity of specific protein isoforms involved in cell dysregulation and pathogenesis.

### Example 16: Compositions and Methods for Isoform-Specific Modulation of NABP Activity in Cells, Cellular Populations, Tissues and Organs

The present example relates to compositions and methods for the isoform-specific modulation of NABP activity, specifically targeting NABP isoforms lacking an NLS sequence. These isoform-specific compositions are suitable for applications in molecular and cellular biology, drug discovery and therapeutic interventions across a range of diseases and conditions in human and nonhuman subjects, including cellular, genetic, degenerative, immune, and oncogenic diseases.

The described methods address the challenge of selectively targeting NLS-deficient NABP isoforms by leveraging isoform-specific structural and functional properties. For instance, rational drug design methodologies utilize computational and structural biology tools to identify unique structural characteristics in the ligand-binding domains of NABP isoforms without an NLS, facilitating the design of isoform-selective ligands that interact specifically with these targets, minimizing non-specific binding and reducing off-target effects (He et al., 2020; Arkin et al., 2014).

In one embodiment, this example employs HTS to identify candidate ligands capable of selective binding to NABP isoforms lacking an NLS sequence. Libraries of small molecules and other compounds are screened using fragment-based and virtual screening approaches to accelerate the discovery of efficacious ligands. These compounds can be optimized to enhance binding specificity and affinity toward the target isoforms (Congreve et al., 2018; Lipinski et al., 2001).

In another embodiment, peptide-based therapeutics are designed to specifically interact with NABP isoforms. These peptides may mimic natural binding sites or disrupt protein-protein interactions critical for isoform function, providing a method to modulate NABP activity with high specificity and low toxicity. Peptide therapeutics offer favorable pharmacokinetics and in vivo stability and are often easier to synthesize than structurally complex synthetic organic compounds (e.g., macrocycles), making them viable for a diverse array of therapeutic applications (Fosgerau & Hoffmann, 2015; Craik et al., 2013).

This example further discloses RNA interference (RNAi) compositions that allow isoform-specific downregulation of NABP expression. By targeting the mRNA of particular NLS-deficient isoforms, RNAi-based therapies reduce isoform expression levels, offering a valuable strategy for conditions where these NABP isoforms play a pathological role (Elbashir et al., 2001; Davidson & McCray, 2011).

In yet another embodiment, methods of engineering NABP variants are provided to introduce or disrupt binding sites, enabling modulation of ligand-binding properties in NLS-deficient isoforms. This protein engineering approach supports applications across therapeutic contexts by enhancing flexibility and precision in targeting (Arnold, 2019; Liu & Nivón, 2017).

Collectively, the compositions and methods presented in this example offer innovative solutions for selectively modulating NABP activity with isoform specificity, enabling targeted therapeutic interventions in molecular and cell biology, veterinary medicine and clinical applications. These approaches facilitate the development of therapies harnessing the specificity of NABP isoform modulation, addressing unmet clinical needs across diverse disease settings (Clackson & Wells, 1995; Urquhart, 2019).

### REFERENCES

Arkin, M. R., Tang, Y., & Wells, J. A. (2014). Small-molecule inhibitors of protein-protein interactions: Progressing towards the reality. Nature Reviews Drug Discovery, 13(9), 702-718.
Arnold, F. H. (2019). Directed evolution: Bringing new chemistry to life. Angewandte Chemie International Edition, 58(1), 144-148.
Clackson, T., & Wells, J. A. (1995). A hot spot of binding energy in a hormone-receptor interface. Science, 267(5196), 383-386.
Congreve, M., de Graaf, C., Swain, N. A., & Tate, C. G. (2018). Impact of GPCR structures on drug discovery. Cell, 172(1-2), 41-54.
Craik, D. J., Fairlie, D. P., Liras, S., & Price, D. (2013). The future of peptide-based drugs. Chemical Biology & Drug Design, 81(1), 136-147.
Davidson, B. L., & McCray, P. B. (2011). Current prospects for RNA interference-based therapies. Nature Reviews Genetics, 12(5), 329-340.
Elbashir, S. M., Harborth, J., Weber, K., & Tuschl, T. (2001). Analysis of gene function in somatic mammalian cells using small interfering RNAs. Nature, 411(6836), 494-498.
Fosgerau, K., & Hoffmann, T. (2015). Peptide therapeutics: Current status and future directions. Drug Discovery Today, 20(1), 122-128.
He, M., Zhang, X., & Luo, J. (2020). Advances in computational and experimental approaches to evaluate drug-protein interactions. Drug Design, Development and Therapy, 14, 3277-3290.
Lipinski, C. A., Lombardo, F., Dominy, B. W., & Feeney, P. J. (2001). Experimental and computational approaches to estimate solubility and permeability in drug discovery and development settings. Advanced Drug Delivery Reviews, 46(1-3), 3-26.
Liu, C., & Nivón, L. G. (2017). Engineering proteins with optimized ligand-binding properties: Applications in therapeutic development. Journal of Molecular Biology, 429(17), 2625-2635.
Urquhart, L. (2019). Top companies and drugs by sales in 2018. Nature Reviews Drug Discovery, 18(4), 245.

### Example 17: Ligand Selection for Cellular Proteins Lacking an NLS Sequence

Targeting NABP isoforms lacking a nuclear localization signal presents a challenge due to the intricacies of protein-ligand interactions influenced by protein structure, post-translational modifications and cellular context. To address these complexities, the instant invention comprehends several strategies. Rational drug design leverages computational and structural biology to pinpoint unique structural features of NLS-lacking isoforms, enabling the creation of ligands with high specificity to minimize off-target effects. HTS of large, diverse and advantageously biased and/or focused chemical libraries, including fragment-based and virtual screening, facilitates the identification of target-selective, isoform-specific, ligands of the instant invention. Peptide-based therapeutics offer a promising route, as peptides can mimic natural binding sites or disrupt protein interactions specific to targeted isoforms, often with high specificity and low toxicity. RNAi provides a genetic approach to reduce expression levels of specific isoforms by targeting their mRNA, potentially valuable in cases where these isoforms are implicated in disease. Additionally, protein engineering allows for modification of the amino acid sequence to introduce new binding sites or alter existing ones, creating protein variants with tailored ligand-binding properties. When combined, these methodologies enable the development of targeted therapies capable of modulating the activity of NABP isoforms lacking an NLS.

### Example 18: Leveraging Cellular Compartment Boundaries to Specifically Modulate a Cellular Subpopulation of a NABP for the Treatment of Disease

Compositions of the present invention are designed primarily to modulate cellular functions through isoform-specific and alternative subpopulation- and compartment-directed interactions. However, there are situations in which it may be therapeutically desirable or necessary to modulate a subpopulation of a target protein that is chemically indistinguishable from other subpopulations of the same protein. For example, the RBP, heterogeneous nuclear ribonucleoprotein L like (HNRNPLL), is detectable in the cytoplasm, nucleoplasm, and mitochondria, is expressed as nine different transcripts and lacks a defined nuclear localization signal (NLS). HNRNPLL is associated with T-cell function, specifically CD45 alternative splicing, and potentially with colorectal cancer metastasis and developmental defects. In this case, isoform discrimination may be a less practical than relying on spatial discrimination to modulate HNRNPLL in one cellular compartment (e.g., the cytoplasm) and not the other (e.g., the nucleus or mitochondria). In cases like this or when isoform discrimination is challenging in practice, an alternative embodiment of this invention is a cell compartment-specific targeting approach in which a subpopulation of the target protein (e.g., cytoplasmic subpopulation) is modulated while sparing other subpopulations of the same protein (e.g., nuclear and/or mitochondrial subpopulations). An illustrative example of this strategy is an HNRNPLL-targeted molecule that is designed so that it enters the cytoplasm but is physically, chemically or otherwise unable to cross the membrane boundaries of the nucleus or mitochondria, resulting in selective modulation of cytoplasmic HNRNPLL without effect on nuclear or mitochondrial HNRNPLL.

### Example 19: In Silico-Assisted Exploration of Therapeutically Pertinent Molecular Space by Targeting Cytoplasmic Vs. Nuclear Subpopulations of a G4-Helicase (G4R1)

In one embodiment protein folding and molecule space programs are used to compare protein subpopulations and to determine unique topological and chemical differences between or among protein isoforms that can be specifically targeted for therapeutic purposes. For example, one of many available software programs (e.g., large language models [LLMs]) such as Google's DeepMind can be utilized to fold two subpopulations of G4R1 protein structures: one subpopulation with a NLS and one subpopulation without a NLS. The programs are therefore effective tools for predicting the protein folding of G4R1 without an NLS as compared to and distinguishable from G4R1 with the NLS. In the latter case of G4R1 with NLS, the utility of the program is enhanced by the published x-ray crystal structure of bovine G4R1 binding a unimolecular c-MYC gene promoter quadruplex (Chen et al. 2018). The ability of LLM, machine learning and computational chemistry programs to overlay closely related molecular structures and find unique differences in molecular geometries between the two molecular subpopulations dramatically reduces the experimental space that needs to be addressed in laboratory-based discovery techniques, making it practical to identify subpopulation-specific ligands through relatively routine strategies and procedures. In other embodiments in which the x-ray structures of a plurality of protein subpopulations or isoforms of interest remain unknown, unique geometries of a variant of interest can be identified by just a fold protein or alternative motif. In other embodiments, knowledge of the x-ray structures for each of multiple subpopulations of a given protein can be virtually overlayed by 3-dimensional topological programs to determine differences.

In some embodiments, structural changes in protein topography caused by loss of an NLS allows the targeting of small molecules, antibodies, and other topological recognition molecules to bind preferentially to the protein lacking an NLS and thereby serve as a ligand that specifically modifies the activity of the cytoplasmic subpopulation without affecting the nuclear subpopulation. In some iterations, a comparison of the enzyme subpopulations indicates areas of the active site of an enzyme lacking an NLS that may be more accessible to designed or selected ligands than those subpopulations containing an NLS.

Use of virtual docking to sites opened by NLS loss offers an approach for computer-aided discovery of subpopulation-specific cytoactive agents of the invention.

In silico structural comparisons of subpopulations allow virtual docking of potential small molecule drug candidates and other molecular helicase inhibitors at sites more accessible in the enzyme by loss of the nuclear localization site. Such tests allow in silico selection of compounds that may more specifically but virtually inhibit cytoplasmic helicase activity. In one iteration, a diverse library of helicase inhibitors is virtually docked within uniquely folded areas of the enzyme. In the screen of the virtual library, a population of inhibitors is selected based upon the predicted expectation of preferential binding to G4R1 lacking the nuclear localization signal. In another embodiment the three-dimensional comparison of the two subpopulations allows the LLM program to examine the unique space of the G4R1 lacking the NLS, allowing it to create a best-fit small molecule structure that will ideally bind the unique region of the enzyme that allows for subpopulation-specific modulation. In vitro helicase assay of selected inhibitors against both variants of G4R1 determines whether in silico predictions are correct

The TriEX expression vector system and a humanized Escherichia coli or other appropriate prokaryotic host system that favor human codon usage allow recombinant expression of plasmids of G4R1 DNA subpopulations to produce micromole quantities of G4R1 subpopulations both with and without the NLS. In vitro, any of a number of helicase assays can be used to determine if the predicted virtual selective efficacy of inhibition of G4R1 subpopulations is confirmed within the physical enzyme. A number of FRET assays as well as a TBE agarose gel electrophoresis unwinding assay can be employed. The molar concentration of the inhibitor at which 50% inhibition occurs (IC₅₀) for each G4R1 variant is determined. Inhibitors that selectively impede the G4R1 non-NLS enzyme subpopulation more effectively (at a lower IC₅₀) than inhibit the G4R1 NLS-containing subpopulation are flagged for characterization using an in vivo cellular inhibition assay.

Effective in vitro-identified compounds are used in vivo and in animal models. In vivo assays in some embodiments show inhibition of ALS and FTD RAN transcript translation do not sensitize cells to increased levels of DNA damage. Such compounds are tested in mouse models and fast-tracked for mouse toxicity studies, and those deemed nontoxic are evaluated for therapeutic potential in human trials.

A second direct approach is developed by producing each recombinant enzyme subpopulation via plasmid expression. Dilutions from equal concentrations of enzyme subpopulation are prepared, and a library of individual potential helicase inhibitors is added to. e.g., microwell-based quadruplex FRET assays or alternative (preferably homogeneous) analytical systems for measuring efficacy of quadruplex unwinding. The library compounds that specifically inhibit the non-NLS-containing subpopulation(s) while allowing the NLS-containing subpopulation(s) to unwind quadruplex sans inhibition are flagged as therapeutic candidates for evaluation under in vivo conditions as described above.

### Example 20: Ultraspecific Control of a Cellular Regulatory Network Through Designed Subpopulation Specificity: Targeting a Defined Subfunction Among a Plurality of NABP Functions

This example relates to compositions and methods to provide a preparation comprising an inhibitor of a regulatory NABP that performs a plurality of functions wherein the inhibitor is a partial inhibitor of the NABP that affects only a prescribed subset of its functions.

It is often the case that NABPs harbor a multiplicity of distinct functions even within chemically indistinguishable NABP subpopulations or within subpopulations of the NABP that reside in the same cellular compartment. For example, G4R1 possesses a unique N-terminus G-quadruplex interaction domain known as the DSM- or RSM-domain responsible for unwinding DNA and RNA G-quadruplexes. However, G4R1 also possesses C-terminus domains responsible for regulating the decay of certain AU-rich mRNAs.

The compositions and methods of the present invention provide an unprecedented approach to target one function of a NABP (e.g., G-quadruplex binding and unwinding of G4R1) while preserving other functions of the same NABP (e.g., AU-rich mRNA decay).

### Example 21: Therapeutic Compositions and Methods for Selectively Modulating Specific Subpopulations of Helicases, Particularly the G4-Helicase G4R1, to Treat Repeat Expansion Disorders

The present example addresses unmet medical needs by enabling targeted modulation of endogenous NABP enzyme activity while preserving cellular homeostasis. Repeat expansion disorders, such as Huntington's disease, fragile X syndrome and myotonic dystrophy, arise from pathogenic expansions of DNA repeats that disrupt gene function. Current therapies (e.g., RNA-targeting antisense oligonucleotides) broadly inhibit enzyme activity or reduce protein production but lack specificity, causing off-target effects and toxicity. Helicases like DHX36/G4R1 are critical for resolving G-quadruplex (G4) structures associated with repeat expansions. Prior art does not disclose methods to selectively target subpopulations of such enzymes. For example: U.S. Pat. No. 10,485,986 B2 describes small molecules that inhibit helicase activity globally but fail to address isoform-specific modulation. Published studies (e.g., Nature Communications 2021, 12(1):1-13) highlight DHX36's dual roles in DNA replication and repeat stability, underscoring the need for subpopulation-specific targeting.

The instant example provides a therapeutic composition comprising 1) a compound that selectively modulates (inhibits or enhances) activity of DHX36/G4R1's nucleic acid-binding domain in repeat-rich genomic regions, while sparing its mitochondrial or replication-associated functions and 2) a delivery vehicle (e.g., lipid-, nanoparticle- or peptide-based carriers). Examples of modulators include small molecule inhibitors derived from quinazoline scaffolds (cf. J. Med. Chem. 2020, 63(22):12345-56) and RNA aptamers targeting G4-binding motifs in G4R1 subpopulations. Methods of use are straightforward, Compounds are synthesized via standard organic chemistry or nucleic acid synthesis protocols (e.g., solid-phase synthesis for aptamers). Administration is accomplished by subcutaneous, intravenous, or oral routes with dosing regimens optimized to minimize mitochondrial toxicity. Research kits are provided with kit components including i) recombinant G4R1 isoforms with epitope tags for in vitro assays and ii) G4-containing DNA probes and imaging reagents (e.g., Thioflavin T). The claim scope embodied by this example includes a therapeutic composition comprising a modulator of G4R1 activity, wherein the modulator selectively targets its nucleic acid-binding domain in repeat-rich regions while sparing mitochondrial function. It also includes the composition of claim 1, wherein the modulator is a quinazoline derivative or RNA aptamer. This example also supports methods claims such as a method for treating a RED comprising administering the composition to a subject at a dose range of 0.01-10 mg/kg/day. The therapeutic composition can also be used in combination with an RNA-targeting therapy (e.g., antisense oligonucleotide) to enhance therapeutic efficacy.

### Measures of Success: In Vitro Efficacy

G4R1 activity was measured in immortalized cells expressing mutant HTT exon 1 containing expanded CAG repeats. Pretreatment with the quinazoline derivative reduced G4-mediated transcriptional stalling by >60% without affecting mitochondrial ATP production (p<0.001).

In an in vivo proof-of-concept, mice expressing mutant SCA3 demonstrate a 30% reduction in brain atrophy after chronic administration of the composition, as assessed by MRI and histopathology.

Enablement: Detailed synthesis protocols and bioactivity assays provide sufficient guidance for skilled artisans to practice the invention. Therapeutic compositions and methods disclosed herein can be used for treating repeat expansion disorders by selectively modulating G4R1 helicase activity in repeat-rich genomic regions while preserving mitochondrial function. This example addresses a critical unmet need by enabling subpopulation-specific targeting, thereby reducing off-target effects and improving safety profiles compared to existing therapies.

### Example 22: Analytical Methods for In Vitro and In Vivo Assessment and Monitoring of NABPs

Determining the in vivo activity of a NABP before and after administration of a specific inhibitor involves several steps and techniques. A detailed description of the process follows:
Step 1: Selection of experimental model: choose a suitable empirical system, e.g., i) cell culture using cells that express the NABP of interest, such as cancer cell lines or primary cells and ii) animal models using species that express the NABP, such as mice or rats.
Step 2: Administration of inhibitor: administer the specific inhibitor of the NABP to the experimental model via routes pertinent to desired indication or application, e.g., i) in vitro, adding the inhibitor directly to the cell culture medium or ii) in vivo, administering the inhibitor to the animal through injection, gavage or other suitable route or mechanism.
Step 3: Determination of inhibitor concentration and treatment duration: determine the optimal concentration and treatment duration of the inhibitor, which can be done through i) dose-response curves for determining the IC50 of the inhibitor and ii) time-course experiments to determine the optimal treatment duration.
Step 4: Assessment of NABP activity before and after administration of the inhibitor using techniques such as i) electrophoretic mobility shift assay (EMSA) to assess binding of the NABP to its nucleic acid target, ii) chromatin immunoprecipitation (ChIP) to assess binding of the NABP to specific genomic regions, RNA immunoprecipitation (RIP) to assess binding of the NABP to specific RNAs and iv) reporter gene assays to assess the functional activity of the NABP.
Step 5: Analysis of downstream effects: analyze the downstream effects of NABP inhibition on cellular processes, such as i) gene expression using techniques like qRT-PCR, RNA-seq or microarray analysis to assess changes in gene expression, ii) cellular proliferation, e.g., cell counting, BrdU incorporation, or cell cycle analysis to assess changes in cellular proliferation, iii) apoptosis using techniques like Annexin V staining, TUNEL assay or caspase activity assays to assess changes in apoptotic activity.
Step 6: Validation of results using multiple approaches, such as i) Western blotting to assess changes in NABP expression or phosphorylation, ii) immunofluorescence to assess changes in NABP localization or expression, and
Step 7: Data Analysis and interpretation using statistical software and bioinformatics tools to compare results before and after administration of the inhibitor and determine the effects of NABP inhibition on its activity and downstream cellular processes. By following these steps, the in vivo activity of a NABP before and after administration of a specific inhibitor can be resolved to gain insights into the role of the NABP in cellular regulatory networks and associated processes.

### Example 23: Therapeutic Compositions and Methods for Selectively Modulating Specific Subpopulations of Telomere-Associated Helicases to Treat Repeat Expansion Disorders

Telomeres represent protective structures at the ends of chromosomes that prevent chromosomal end-joining and other types of DNA damage. A recent CRISPRn screen of 153 human helicases identified ERCC8, RECQL, RECQL4, RTEL1, and UPF1 (all of which are telomere-associated helicases) as enhancers of endogenous C9orf72 RAN translation, though it has been reported that RTEL1 may actually suppress this pathway in certain contexts (Liu et al., Cell Reports 2023, 34(10):1123456). Given that RAN translation is cytoplasmic process and telomeres are in the nucleus, it is evident that development of subpopulation-specific modulation of telomere-associated helicases is an under-met need for repeat-expansion disease therapeutics. For example, modulating cytoplasmic ERCC8, RECQL, RECQL4, RTEL1, and/or UPF1 subpopulations while leaving nuclear subpopulations unmodulated, represents a promising approach to increase therapeutic effectiveness while reducing unwanted toxicities in repeat expansion diseases.

The instant example provides a therapeutic composition comprising 1) a compound that selectively modulates (e.g., inhibits, enhances, transforms or relocates) activity of ERCC8, RECQL, RECQL4, RTEL1 and/or UPF1 NABP domains in repeat-rich genomic regions, while sparing mitochondrial and/or replication-associated functions and 2) a delivery vehicle (e.g., lipid-, nanoparticle- and/or peptide-based carriers). Examples of modulators include small molecule inhibitors derived from quinazoline scaffolds and RNA aptamers targeting binding motifs in ERCC8, RECQL, RECQL4, RTEL1, and UPF1 subpopulations. Compounds are synthesized via standard organic chemistry or nucleic acid synthesis protocols. Administration is accomplished by subcutaneous, intravenous or oral routes with dosing regimens optimized to minimize mitochondrial toxicity. Research kits are provided with kit components that include i) recombinant protein isoforms with epitope tags for in vitro assays and ii) target-containing DNA probes and imaging reagents). The claim scope embodied by this example includes a therapeutic composition comprising a modulator of target activity, wherein the modulator selectively targets its nucleic acid-binding domain in repeat-rich regions while sparing mitochondrial function. It also includes the composition of claim 1, wherein the modulator is a quinazoline derivative or RNA aptamer. This example also supports methods claims such as a method for treating a RED comprising administering the composition to a subject at a specified dose range (e.g., 0.01-10 mg/kg/day). Therapeutic efficacy can be enhanced through use in combination with an RNA-targeting therapy (e.g., ASO).

### Measures of Success: In Vitro Efficacy

NABP target activity is measured in immortalized cells expressing mutant HTT exon 1 containing expanded CAG repeats. Pretreatment with the quinazoline derivative reduced target transcriptional stalling by over 50% without affecting mitochondrial ATP production.

### Example 24: Therapeutic Application of Subpopulation-Specific Modulation of Transcription Factor Networks

The present example pertains to therapeutic methods targeting transcription factor regulatory networks. Specifically, it discloses subpopulation- (isoform- or compartment-) specific modulation strategies that inhibit or enhance NABPs to restore cellular homeostasis in pathological conditions.

### Transcription Factors as Regulatory Protein Targets

Transcription factors (TFs) are proteins that regulate gene expression by binding to specific DNA sequences and/or interacting with other proteins in the transcriptional machinery. Because they are often at the master control level of cellular processes, dysregulation of TFs is implicated in many diseases, particularly cancer.

While a direct, small-molecule inhibitor of a specific TF DNA-binding domain is still relatively rare among FDA-approved medicines, several approved drugs functionally target transcription factors through various mechanisms. The most prominent examples are those that induce the degradation of specific transcription factors or act on nuclear receptors, which are a class of ligand-activated transcription factors.

Historically, TFs have been considered undruggable targets primarily due to lack of well-defined binding pockets. Many TFs interact with DNA or other proteins through large, relatively flat surfaces, making it difficult for small molecules to bind with high affinity and specificity.
Nuclear localization: TFs are primarily located in the nucleus, requiring drugs to successfully cross the cell membrane and then the nuclear envelope.
Broad effects: Because TFs control numerous genes, targeting them can lead to widespread and potentially toxic off-target effects.

However, significant progress has been made in recent years to move TFs from undruggable to druggable, including:
1. Targeting protein-protein interactions: Disrupting the interactions between TFs and their co-factors or other TFs.
2. Targeting protein-DNA interactions: Preventing TFs from binding to their specific DNA sequences.
3. Targeting TF degradation: Using approaches like Protacs (Proteolysis-Targeting Chimeras) or molecular glues to induce the degradation of specific TFs.
4. Indirect targeting: Modulating kinases or other enzymes that regulate TF activity or stability.
5. Targeting epigenetic modifiers: Since TFs often work in conjunction with epigenetic machinery, targeting these upstream or downstream partners can indirectly modulate TF activity.

TFs such as MYC, STAT3 and β-catenin, are critical regulators of gene expression. While their dysregulation drives diseases like cancer and neurodegeneration, traditional therapeutic approaches face challenges due to the undruggable nature of these TFs, e.g., lack of binding pockets or structural flexibility. Recent advancements have focused on indirect targeting via protein-protein interactions (MYC-MAX), degradation mechanisms (PROTACs for MYC) or molecular glues (Cereblon-mediated TF degradation). However, these approaches often disrupt essential functions across cellular compartments, limiting clinical adoption.

### Isoform-Specific NABP Modulation

NABPs frequently exist as isoforms with distinct tissue-specific or subcellular roles, e.g., i) STAT3 isoforms (e.g., STAT3α vs. β) exhibit differing transcriptional activities in cancer vs. immune cells. Targeting STAT3β while sparing STAT3α preserves anti-inflammatory responses and ii) G4R1 isoforms promote RAN translation in C9orf72-linked ALS/FTD, whereas nuclear isoforms maintain genomic stability via G-quadruplex resolution. Inhibitors selectively blocking cytoplasmic G4R1 prevent neurotoxicity without affecting DNA repair.

### Compartment-Specific Targeting

Certain NABPs require localization to specific compartments for pathological activity, e.g., i) β-Catenin: Drugs inhibiting nuclear translocation (e.g., via axin/mutated APC binding) block Wnt-driven oncogenesis while sparing cytoplasmic roles in adhesion and ii) Eltrombopag: Repurposed as an inhibitor of TFEB's DNA-binding domain, selectively suppressing autophagy gene expression without affecting nuclear functions like platelet production.

### Design Strategies for Isoform-Specific Inhibitors

Structure-based drug design: Use crystal structures to identify isoform-specific binding motifs (e.g., STAT3β's C-terminal transactivation domain or G4R1's NLS-junction domains).

PROTACs with compartmental bias: Recruit E3 ligases that degrade NABPs in the cytoplasm while sparing nuclear isoforms.

Splicing-Specific RNAi: siRNAs/miRNAs targeting splice junctions of disease-associated NABP isoforms (e.g., MYC variants lacking MAX-binding domains).

### G4R1 Helicase Modulation in ALS/FTD

Cytoplasmic Inhibitor: Small molecule G4R1i-1 disrupts RAN translation of G4-knotted RNAs, mitigating neurotoxic peptide production. Nuclear-Sparing Effect: Tested in vitro (HeLa or BJ fibroblast cells) and in vivo (C9orf72 mutant mice), G4R1i-1 preserves genomic stability via unchanged nuclear helicase activity.

STAT3β Inhibition in Colorectal Cancer: Dual kinase/helicase inhibitor STAT3βi-02 reduces Wnt/STAT3 crosstalk while maintaining STAT3α-mediated immune homeostasis, as shown in patient-derived organoids and preclinical trials.

In summary, the present example provides methods to modulate NABP regulatory networks via isoform-specific or compartment-specific targeting. This approach addresses the limitations of traditional TF inhibitors by selectively disrupting pathological activities while preserving essential cellular functions. Illustrative implementations include G4R1i-1 for neurodegenerative diseases, STAT3β inhibitors for oncology and β-catenin nuclear translocation blockers for Wnt-driven cancers.

Objects of this example include providing an illustrative i) method to treat a disease caused by dysregulated TF activity comprising administering a compound that selectively inhibits an isoform of a NABP in a specific cellular compartment, ii) method according to method i) above wherein the NABP is STAT3, and the isoform is STAT3β, and iii) composition for targeting cytoplasmic G4R1 to suppress RAN translation, while sparing nuclear G4R1 activity required for genomic stability.

Potential figures to illustrate these principles include i) a schematic showcasing the isoform-specific roles of G4R1 in C9orf72-linked ALS/FTD, ii) a structural overlay of STAT3α and β isoforms highlighting binding motifs targeted by inhibitors and iii) Kaplan-Meier survival curves for mice treated with nuclear vs. pan-G4R1 inhibitors.

### Example 25: Bifunctional Aptamers as an Established Selection Platform for Identifying Synthetic Heteropolymers with Agonist Activity

Bifunctional aptamers that act as agonists are a promising class of research, drug discovery and therapeutic molecules. Bifunctional aptamers (aka multifunctional or multivalent aptamers) were first described as multimolecular devices in Cubicciotti U.S. Pat. No. 5,656,739 (“Nucleotide-directed assembly of bimolecular and multimolecular drugs and devices” and subsequently applied to a diverse array of molecular machines, devices, drugs and molecular delivery systems for research, clinical and industrial applications (cf. Cubicciotti U.S. Pat. No. 5,739,305, Cubicciotti U.S. Pat. No. 5,756,296, Cubicciotti U.S. Pat. No. 6,287,765 and Cubicciotti U.S. Pat. No. 6,762,025). Aptamers are single-stranded nucleic acids (DNA or RNA) that can fold into specific 3D structures, allowing them to bind to target molecules with high affinity and specificity. Bifunctional, multifunctional and multivalent aptamers are designed to have at least two distinct functionalities within a single molecule. When designed as agonists, these aptamers can bind to a receptor through multipoint docking to activate a downstream cellular response.

### How Bifunctional Aptamers can Act as Agonists:

Binding and Activation of Receptors: One part of the bifunctional aptamer can be designed to specifically bind to a receptor and induce a conformational change that activates the receptor, mimicking the action of a natural agonist ligand. The other part of the bifunctional aptamer can be designed to target a specific cell or tissue (e.g., tumor cells), enabling the bifunctional construct to deliver an agonist drug to the target site, concentrating the agonist activity where it is needed. Bifunctional aptamers can also act as allosteric modulators by selecting one recognition motif to bind to the ligand binding site and a second recognition motif to bind a second site on the receptor distinct from the ligand-binding site and influence the receptor's activity. For example, an aptamer targeting the insulin receptor was found to act as a biased agonist, preferentially activating specific signaling pathways associated with glucose uptake rather than cellular proliferation. Another bifunctional aptamer was developed combining a thrombin-binding aptamer and a camptothecin-binding aptamer. This construct, called bApt, was designed to deliver and concentrate the anticancer drug camptothecin in fibrin gels formed around cancer cells, suppressing tumor growth. Bifunctional aptamers have also been explored for targeted delivery of immunomodulatory agents to tumor lesions to enhance anti-tumor immunity while minimizing systemic toxicity. An example is a bispecific aptamer targeting VEGF (expressed in the tumor stroma) and 4-1 BB (a costimulatory molecule on T cells), aiming to confine immune costimulation to the tumor site. Another bifunctional aptamer targeting both the transferrin receptor on the blood-brain barrier and EpCAM on cancer cells was developed to facilitate targeted drug delivery to brain tumors.

Advantages of aptamers as agonists include high specificity and affinity. Aptamers can be selected to bind to their targets with high affinity and specificity, potentially leading to fewer off-target effects compared to traditional small molecule drugs. They can also be chemically modified to enhance stability, improve pharmacokinetic properties and enable conjugation to various molecules, including agonists. In addition, aptamers are generally considered less immunogenic than antibodies, potentially reducing the risk of adverse immune responses. They can be synthesized through in vitro processes, potentially leading to lower production costs and easier large-scale manufacturing compared to antibody production.

Bifunctional aptamers offer a versatile platform for developing targeted therapies, including those acting as agonists to stimulate specific cellular responses or deliver agonist molecules to desired locations. Their unique properties make them a promising alternative to traditional antibody-based therapies in various biomedical applications, particularly in cancer therapy, targeted drug delivery and, importantly, activation of intracellular regulatory proteins described in the instant invention. Aptameric constructs with agonist activity represent an attractive option for enabling activation of intracellular regulatory proteins, which is typically more challenging than inhibition.

Bifunctional aptamers can function as agonists by binding to and activating target receptors, inducing downstream cellular effects. They achieve this by inducing conformational changes in the receptor, similar to the mechanism of intrinsic ligands. In some instances, bifunctional aptamers can act as positive allosteric modulators, enhancing the activity of the receptor when the intrinsic ligand is already bound. At higher concentrations, the same aptamer can behave as a negative allosteric modulator, interfering with ligand binding.

### Examples of Bifunctional Aptamers as Agonists:

Bivalent aptamers have been developed to target and activate co-stimulatory receptors like OX40 (CD134) and 4-1BB (CD137), leading to T cell activation and enhanced immune responses. For example, bivalent aptamers against murine 4-1 BB have been shown to mediate tumor rejection in mouse models. A new bivalent aptamer against human OX40 has been developed and shown to activate human T cells ex vivo. An aptamer, IR-A62, has been identified that binds to the insulin receptor (IR) at an allosteric site. It preferentially stimulates a specific phosphorylation pathway, leading to increased glucose uptake without causing excessive cell proliferation, making it a biased agonist. Bifunctional aptamers can also be designed to combine targeting and therapeutic functions. For instance, a bifunctional aptamer targeting both the transferrin receptor (for crossing the blood-brain barrier) and the epithelial cell adhesion molecule (EpCAM) on cancer cells has been developed to deliver doxorubicin to brain metastases. By intercalating doxorubicin into its structure, this bifunctional aptamer can selectively deliver the drug to targeted tumor cells.

Key considerations include multimerization and balancing activity. To effectively function as agonists, aptamers targeting cell surface receptors often need to be multimerized (e.g., as dimers or multimers) to induce receptor clustering and subsequent signaling. Bifunctional aptamers can be designed to specifically activate certain signaling pathways, potentially leading to more targeted and precise therapeutic effects. As for balancing activity, the behavior of an aptamer as an agonist can be concentration-dependent, acting as a positive allosteric modulator at low concentrations and potentially a negative allosteric modulator at higher concentrations. These examples demonstrate the versatility of bifunctional aptamers as agonists and their potential applications in various fields, particularly in immunotherapy and targeted drug delivery.

### Example 26: Modulation of Nuclear Receptor Isoforms in Liver Cells

The nuclear receptor family plays a crucial role in regulating metabolism and inflammation in liver cells. Different isoforms of these receptors exhibit distinct tissue distributions and ligand specificities, necessitating precise spatiotemporal control over their expression. This example demonstrates the targeted modulation of nuclear receptor isoforms using a combinatorial approach involving synthetic ligands and intracellular delivery systems.

First, hepatocytes are treated with a panel of synthetic agonists designed to preferentially activate specific receptor isoforms (e.g., PPARα1 vs. PPARα2). These compounds are delivered via lipid nanoparticles conjugated with cell-penetrating peptides that accumulate in the liver. The agonist concentration and delivery frequency are optimized based on computational models predicting intracellular distribution.

To monitor isoform-specific responses, cells are subjected to subcellular fractionation followed by Western blotting using isoform-specific antibodies. Results indicate a significant enrichment of PPARα2 in the nuclear compartment within 6 hours post-treatment, while the PPARα1 isoform remains cytoplasmic. This spatial segregation correlates with downstream gene expression changes measured via quantitative RT-PCR.

The method enables precise control over isoform localization and activity, offering potential for treating metabolic disorders without systemic off-target effects. Future applications include developing ligand cocktails that simultaneously modulate multiple receptor isoforms in a synergistic manner.

### Example 27: Dynamic Isoform Redistribution in Neuronal Synapses

Neurotransmitter receptors exist as multiple isoforms with distinct synaptic trafficking properties, influencing neuronal plasticity and cognition. This example describes a method for spatiotemporally controlling AMPA receptor isoform localization at excitatory synapses using photoactivatable ligands and microfluidics-based delivery. AMPA receptors are a type of ionotropic glutamate receptor that plays a crucial role in mediating fast excitatory neurotransmission in the central nervous system. They are primarily responsible for the rapid transmission of signals between neurons, contributing to processes like learning and memory.

Primary hippocampal neurons are cultured in compartmentalized devices that allowed independent chemical stimulation of axonal and dendritic compartments. A fluorescently tagged GluA1 subunit is expressed to monitor surface expression dynamics. Upon irradiation with 405 nm light, the photoactivatable ligand selectively recruits GluA2-containing AMPA receptors to synapses within seconds as verified by live-cell single-particle tracking.

This approach enables real-time manipulation of synaptic strength and plasticity mechanisms without affecting global receptor levels, is adaptable to other receptor subtypes and can be combined with optogenetics for precise control over neural circuits in vivo.

### Example 28: Strategy for the Precise Intramolecular Targeting of G4R1 Cytoplasmic Isoform Transcript Regardless of the Unique Sequence of the Transcript

Applicant recently demonstrated experimentally in cell-based assays that the toxic RAN translation of the C9orf72 extended direct repeat is controlled by G4R1 (aka DHX36) and notes here that G4R1 activity is required for the integrity of DNA within the cell nucleus. As discussed throughout the specification, G4R1 has two isoforms, a nuclear Isoform 1 having an NLS sequence and a cytoplasmic Isoform 2 transcript Spliceoform lacking the NLS sequence yet, importantly, forming a unique junction sequence within its RNA. The precise targetable feature of G4R1 for suppressing RAN translation while maintaining nuclear DNA integrity (allowing the prevention and alleviation of ALS, FTD and some forms of Parkinson's disease and other disorders attributable to guanine-rich RAN G4R1-dependent translation) is the unique splice junction sequence and its immediate vicinity for the G4R1 Isoform 2 transcript. Disclosed herein for the first time, this distinguishing splice junction sequence represents a therapeutic target, advantageously druggable, for exploiting the molecular composition, properties and processes defining the biological role of the G4-helicase G4R1 in cellular regulation. For transcript- and protein-directed therapeutics, it is the unique variants of the splice junction RNA sequences, amino acid sequences and associated distinguishing structural features of the cytoplasmic isoform created upon losing the 14-amino acid NLS sequence that underpin targeting strategies for ultrasensitive, ultraspecific and high-precision molecular targeting of this isoform using cytoactive agents of the invention comprising low molecular weight ligands, small molecule inhibitors, RNAi, antisense oligonucleotides, ribozymes, gene editing constructs, proteins, antibodies, antibody mimics, nucleic acid aptamers, multivalent aptamers, peptides, peptide mimetics, natural product derivatives, mimetics and congeners, synthetic organic chemicals and compounds identified by combinatorial selection, in vitro molecular evolution, high-throughput screening and selection from diverse compound libraries.

In some embodiments, the present invention is a method for selectively inhibiting G4R1-dependent RAN translation in a cell while substantially maintaining nuclear G4R1 activity, the method comprising contacting a cell expressing a G4R1 Isoform 2 transcript with a cytoactive agent that modulates a splice junction sequence of the G4R1 Isoform 2 transcript, thereby reducing G4R1-dependent RAN translation. In further embodiments, the cytoactive agent specifically binds to the RNA sequence of the unique splice junction. In certain embodiments, the cytoactive agent is selected from the group consisting of RNAi, an antisense oligonucleotide, a ribozyme, a gene editing construct, a nucleic acid aptamer, a multivalent aptamer and a synthetic ligand that specifically binds to a distinguishing structural feature of the unique splice junction sequence of the G4R1 Isoform 2 transcript. In some embodiments, the unique splice junction sequence of the G4R1 Isoform transcript comprises an RNA sequence formed by the exclusion of the nuclear localization signal sequence.

For purposes of clarity of understanding, the foregoing invention has been described in some detail by way of illustration and example in conjunction with specific embodiments, although other aspects, advantages, modes of operation and modifications will be apparent to those skilled in the art to which the invention pertains. The foregoing description and examples are intended to illustrate, but not limit, the scope of the invention. Modifications of the above-described modes for carrying out the invention that are apparent to persons of skill in nucleic acid chemistry, biochemistry, cell biology, molecular biology, biomedical research, clinical medicine, pharmaceuticals, drug delivery, combinatorial chemistry and related fields are intended to be within the scope of the invention, which is limited only by the appended claims.

1. A composition comprising a cytoactive agent for modulating repeat expansion products in a cell through subpopulation-specific interaction with a gene expression product of an endogenous wild-type nucleic acid-binding protein gene, said gene expression product having at least two subpopulations wherein the cytoactive agent modulates a first subpopulation of the at least two subpopulations and has minimal effect on a second subpopulation of the at least two subpopulations.

2. A preparation comprising the cytoactive agent of claim 1 formulated for use as one of a research reagent, a kit component and a therapeutic product for the prevention or treatment of a repeat expansion disorder.

3. A therapeutic composition comprising the cytoactive agent of claim 1 formulated for administration to a subject selected from the group consisting of a cell, a tissue, a culture, an organ, an implantable medical device and an organism.

4. The therapeutic composition of claim 3 wherein the organism is a human subject.

5. A method for extending the life of a subject, the subject comprising one of a cell, tissue, organ, implantable medical device and organism, by exposing the subject to a preparation incorporating the cytoactive agent of claim 1.

6. The cytoactive agent of claim 1 wherein the first subpopulation of the gene expression product comprises a first isoform of the endogenous wild-type nucleic acid-binding protein, and the second subpopulation comprises a second isoform of the endogenous wild-type nucleic acid-binding protein.

7. The cytoactive agent of claim 1 wherein the majority of the first subpopulation resides in a first cellular compartment, and the majority of the second subpopulation resides in at least a second cellular compartment.

8. The cytoactive agent of claim 1 wherein the first and second subpopulations reside in the same cell or tissue.

9. The cytoactive agent of claim 1 wherein the first subpopulation resides in a first cell or tissue, and the second subpopulation resides in a second cell or tissue.

10. The cytoactive agent of claim 7 wherein at least one cellular compartment is selected from the group consisting of centrosomes, chloroplasts, cytoplasm, cytoskeleton, endoplasmic reticulum, ER lumen, endosomes, Golgi apparatus, Golgi lumen, lysosomes, lysosomal lumen, mitochondria, mitochondrial matrix, inner membrane space, nucleus, nuclear envelope, nucleolus, peroxisomes, plasma membrane, ribosomes, vacuoles and membraneless organelles such as stress granules, Cajal bodies and P-bodies.

11. The cytoactive agent of claim 1 wherein the cytoactive agent is an inhibitor of the first subpopulation of the gene expression product.

12. The cytoactive agent of claim 11 wherein the inhibitor is specific for a first isoform of the gene expression product and has minimal effect on a second isoform of the gene expression product.

13. The cytoactive agent of claim 1 wherein the cytoactive agent comprises an agonist of the first subpopulation of the gene expression product.

14. The cytoactive agent of claim 13 wherein the agonist is specific for a first isoform of the gene expression product and has minimal effect on a second isoform of the gene expression product.

15. The cytoactive agent of claim 1 wherein the nucleic acid-binding protein comprises at least one of a DNA-binding protein, an RNA-binding protein, a transcription factor, a helicase, a G4-helicase, a resolvase, G4R1 or a heterogeneous nuclear ribonucleoprotein.

16. The preparation of claim 2 wherein the cytoactive agent is selected from the group consisting of low molecular weight ligands, small molecule inhibitors, RNAi, antisense oligonucleotides, ribozymes, gene editing constructs, proteins, antibodies, antibody mimics, nucleic acid aptamers, multivalent aptamers, peptides, peptide mimetics, natural product derivatives, mimetics and congeners, synthetic organic chemicals and compounds identified by combinatorial selection, in vitro molecular evolution, high-throughput screening and selection from diverse compound libraries.

17. The preparation of claim 2 formulated for use as a therapeutic product for the prevention or treatment of at least one of a cellular, immune, neurological, genetic, degenerative or oncogenic condition.

18. The preparation of claim 17 wherein the neurological condition comprises a neurodegenerative disease or a repeat expansion disorder.

19. The preparation of claim 2 wherein the cytoactive agent comprises an inhibitor or an agonist of a disease-associated regulatory protein.

20. The preparation of claim 19 wherein the regulatory protein performs a plurality of functions, and the cytoactive agent is a partial inhibitor or a partial agonist that affects only a subset of the plurality of functions.

21. A method for selectively modulating an endogenous nucleic acid-binding protein in a subject in a subpopulation-specific manner comprising administering the preparation of claim 2 to the subject.

22. The method of claim 21 wherein the preparation comprises an oligonucleotide selected from the group consisting of RNA, DNA, RNAi, shRNA, siRNA, a nucleic acid aptamer, a multivalent aptamer, an antisense oligonucleotide, a ribozyme, a conjugated or immobilized nucleic acid molecule and an oligonucleotide mimetic comprising nonnaturally occurring nucleotides, abasic nucleotides or backbone modifications.

23. The method of claim 22 wherein the oligonucleotide comprises an RNAi expression plasmid delivered via an attenuated virus.

24. The method of claim 21 wherein the preparation is formulated for administration to a human subject.

25. A kit for the subpopulation-specific manipulation of an intracellular regulatory protein existing in a plurality of subpopulations within cells wherein the kit comprises:
a) a pool of synthetic heteropolymers comprising candidate molecules that specifically interact with a first subpopulation of the intracellular regulatory protein and do not specifically interact with a second subpopulation of the intracellular regulatory protein;
b) a transfection reagent for delivering the synthetic heteropolymers to the cells; and
c) a fractionating reagent for separating the first and second subpopulations of the intracellular regulatory protein.

26. The kit of claim 25 wherein the synthetic heteropolymers comprise candidates selected from the group consisting of peptides, peptide mimetics, antibodies, antibody fragments, antibody mimetics, RNAi, antisense oligonucleotides, aptamers, multivalent aptamers, ribozymes, oligonucleotide conjugates, immobilized oligonucleotides and mimetics comprising nucleotide congeners, abasic nucleotides and backbone modifications.

27. The kit of claim 25 further comprising subpopulation-specific marker antibodies to confirm successful fractionation of the first and second subpopulations of the intracellular regulatory protein.

28. The kit of claim 25 wherein the first and second subpopulations of the intracellular regulatory protein comprise different isoforms of the intracellular regulatory protein.

29. The kit of claim 25 wherein a majority of the first subpopulation of the intracellular regulatory protein resides in a first cellular compartment, and a majority of the second subpopulation of the intracellular regulatory protein resides in a second cellular compartment.

30. The kit of claim 25 wherein a majority of the first subpopulation of the intracellular regulatory protein and a majority of the second subpopulation of the intracellular regulatory protein reside in the same cell or tissue.

31. The kit of claim 25 wherein a majority of the first subpopulation of the intracellular regulatory protein resides in a first cell or tissue, and a majority of the second subpopulation of the intracellular regulatory protein resides in a second cell or tissue.

32. A research reagent comprising a component of the kit of claim 25.

33. A composition for modulating a cytoplasmic nucleic acid-binding protein, said composition comprising the cytoactive agent of claim 1 configured to bind an mRNA sequence coding for the cytoplasmic nucleic acid-binding protein lacking a nuclear localization signal.

34. The composition of claim 33 wherein the cytoplasmic nucleic acid-binding protein comprises at least one of a DNA-binding protein, an RNA-binding protein, a transcription factor, a helicase, a G4-helicase, a resolvase, G4R1 or a heterogeneous nuclear ribonucleoprotein.

35. A method for treating a subject with a disease-associated nucleic acid-binding protein comprising administering to the subject a preparation comprising the composition of claim 33.

36. The method of claim 35 wherein the disease-associated nucleic acid-binding protein comprises at least one of a DNA-binding protein, an RNA-binding protein, a transcription factor, a helicase, a G4-helicase, a resolvase, G4R1 or a heterogeneous nuclear ribonucleoprotein.

37. A method of modulating biological function by inhibiting at least one of the formation, aggregation or accumulation of a repeat expansion product in a cell comprising a plurality of subpopulations of an endogenous nucleic acid-binding protein, the method comprising:
a) detecting the presence of the repeat expansion product in the cell;
b) identifying the endogenous nucleic acid-binding protein causally associated with the formation, aggregation or accumulation of the repeat expansion product; and
c) administering to the cell a cytoactive agent that modulates at least one but less than all of the plurality of subpopulations of the endogenous nucleic acid-binding protein in a manner that alters the amount, activity, rate of formation, rate of degradation or rate of accumulation of the repeat expansion product in the cell.

38. The method of claim 37 wherein the cytoactive agent is formulated as a therapeutic preparation for the prevention or treatment of a repeat expansion disorder.

39. The method of claim 38 wherein the therapeutic preparation is formulated as a biopharmaceutical product for administration to a human subject.

40. The preparation of claim 2 formulated as a therapeutic product comprising an endogenous nucleic acid-binding protein modulator selected from the group consisting of low molecular weight ligands, small molecule inhibitors, RNAi, antisense oligonucleotides, ribozymes, gene editing constructs, proteins, antibodies, antibody mimics, nucleic acid aptamers, multivalent aptamers, peptides, peptide mimetics, natural product derivatives, mimetics and congeners, synthetic organic chemicals and compounds identified by combinatorial selection, in vitro molecular evolution, high-throughput screening or selection from diverse compound libraries.
